(19) United States
(12) Patent Application Publication
(10) Pub. No.: US 2020/0165308 A1
(43) Pub. Date: May 28, 2020

(54) NETRIN- 1 AND DEPENDENCE RECEPTOR PROTEINS AND METHOD OF USE

(71) Applicant: UNIVERSITY OF MANITOBA, Winnipeg (CA)

(72) Inventor: Jorg Stetefeld, Winnipeg (CA)

(21) Appl. No.: 16/663,080

(22) Filed: Oct. 24, 2019

Related U.S. Application Data
(63) Continuation of application No. 15/117,863, filed on Aug. 10, 2016, now abandoned.
(60) Provisional application No. 61/937,771, filed on Feb. 10, 2014.

(51) Int. Cl. C07K 14/475; C07K 16/18; A61K 39/395; C07K 14/705; C07K 14/47

(52) U.S. Cl. CPC C07K 14/475; C07K 16/18; A61K 39/395; A61K 38/00; C07K 14/47; C07K 2317/34; C07K 2319/00; C07K 14/70503

(57) ABSTRACT

Provided herein are fragments of Netrin-1 proteins, fragments of DCC proteins and NEO1 proteins, and antibodies to epitopes located within the fragments of Netrin-1, DCC, and/or NEO1. Also provided herein are methods for using the fragments and antibodies, including methods for inhibiting binding of Netrin-1 to an UNC5 protein, a DCC protein, and/or a NEO1 protein. Other methods provided herein include inducing apoptosis of a cell, and reducing formation of multimers of Netrin-1 proteins, and treating a subject having a cancer or at risk of having a cancer.

### CROSS-REFERENCE TO RELATED APPLICATIONS

This application is a continuation application of U.S. patent application Ser. No. 15/117,863, filed Aug. 10, 2016, which is the § 371 U.S. National Stage of International Application No. PCT/IB2015/050992, filed 10 February 2015, which claims the benefit of U.S. Provisional Application Ser. No. 61/937,771, filed Feb. 10, 2014, each of which are incorporated by reference herein.

### SEQUENCE LISTING

This application contains a Sequence Listing electronically submitted via EFS-Web to the United States Patent and Trademark Office as an ASCII text file entitled “2019-12-27-Sequence-Listing-3520034 ST25” having a size of 81 kilobytes and created on Dec. 27, 2019. The information contained in the Sequence Listing is incorporated by reference herein.

### BACKGROUND

Netrin-1 (NET1) was discovered in 1994 as a guidance cue promoting commissural axon outgrowth (Serafini et al., Cell 78, 409 (1994)). It is expressed in developing and adult nervous systems, but was also located in developing heart, lung, pancreas, intestine, and mammary gland tissues (Kennedy et al., Cell 78, 425 (1994), Liu et al., Curr Biol 14, 897 (2004), Yebra et al., Dev Cell 5, 695 (2003)). It is an essential mediator of vertebrate development, due to its key role in attraction and repulsion of axons, cell migration, regulation of cell survival, and cellular differentiation. The importance of netrin is further underscored in that knockout mice die within the first day of birth (Serafini et al., Cell 87, 1001 (1996)). NET1 is a cysteine rich glycoprotein containing approximately 600-residues that activates cell surface receptors to initiate multiple downstream signal transduction pathways. The dependence receptors Deleted in Colorectal Cancer (DCC) and its paralogue neogenin (NEO1) are crucial for NET1-mediated axon attraction, cell-cell adhesion and tissue organization. In contrast, binding to UNC5 triggers a chemorepellent response (Hong et al., Cell 97, 927 (1999)). Disruption of the NET1 binding to its dependence receptors induces apoptosis (“dependence receptor hypothesis”) (Mehlen et al., Apoptosis 9, 37 (2004)).

NET1 is a member of the family of secreted netrins, which consists of NET1, NET3, and NET4 in humans. The molecule is composed of an N-terminal domain VI, followed by three laminin-type epidermal growth factor (LE) repeats (V-1, V-2 and V-3) and a positively charged C-terminal domain (domain C). NET1 is homologous to domains VI and V of the laminin-γ1 chain, whereas domain C exhibits sequence similarity to tissue inhibitors of matrix metalloproteinases (TIMPs). Genetic studies suggest that domains VI and V are required for axon guidance, whereas domain C provides a signal that prevents axon branching in C. elegans (Lim et al., J Neurosci 22, 7080 (2002), Wadsworth et al., Neuron 16, 35 (1996)). The dependence receptors NEO1/DCC and UNC5 are both single-pass transmembrane receptors of the Ig superfamily (Winberg et al., Cell 93, 581 (1998), Wang et al., J Neurosci 19, 4938 (1999)). The extracellular NET1 binding segments are composed of N-terminal Ig-tandems followed by six fibronectin type III (FN) domains (NEO1/DCC) or a thrombospondin (TSP) tandem repeat (UNC5). Current structural knowledge is limited to unliganded N-terminal Ig tetramers of DCC which assume a horseshoe configuration and the C-terminal tandem of FN domains five and six (FN[5-6]), respectively (Yang et al., J Struct Biol 174, 239 (2010)). A complex between FN[5-6] and the Repulsive Guidance Molecule (RGM) was recently determined, providing structural insight into signaling hub formation (Bell et al., Science 341, 77 (2013)). Mutagenesis studies on NEO1/DCC identified several potential patches on the FN[5-6] segment that are important for binding to NET1 (Bennett et al., J Biol Chem 272, 26940 (1997),

Geisbrecht et al., J Biol Chem 278, 32561 (2003), Kruger et al., J Neurosci 24, 10826 (2004)). However, the localization of distinct binding epitopes for NET1 remains controversial. Much less is known about the UNC5-NET1 complex formation. Indeed, only the Ig1-2 tandem repeat of UNC5 has been shown to be involved in NET1 binding (Geisbrecht et al., J Biol Chem 278, 32561 (2003)).

### SUMMARY OF THE APPLICATION

Netrin-1 (NET1) is known to play several essential roles in the biology of developing and adult systems, including nervous systems, and is believed to play a role in certain cancers. An important characteristic of NET1 is the ability to bind the dependence receptors Deleted in

Colorectal Cancer (DCC), the DCCs paralogue neogenin (NEO1), and UNC5. Evidence has suggested that the VI domain of NET1 binds these dependence receptors. As reported herein, it has been determined that it is not the VI region that binds the dependence receptors, rather it is the V-2 region of NET1 that binds the dependence receptors. In addition, the region of Neogenin that binds to Netrin-1 has also been identified.

Provided herein are NET1 fragments. In one embodiment, a NET1 fragment includes an amino acid sequence having at least 80% identity to amino acids CNLHARRCRFNMELYKLSGRKSGGVCLN (SEQ ID NO:16) wherein the NET1 fragment has UNC5 binding activity, DCC/NEO1 binding activity, or the combination thereof. In one embodiment, the NET1 fragment includes an UNC5 binding domain HARRCR (SEQ ID NO:9) that includes conservative substitutions at positions 1, 2, 3, 4, 5, 6, or a combination thereof. In one embodiment, NET1 fragment includes a DCC/NEO1 binding domain MELYKLS (SEQ ID NO:10) that includes conservative substitutions in MELYKLS (SEQ ID NO:10) at positions 1, 2, 3, 4, 5, 6, 7, or a combination thereof. In one embodiment, the NET1 fragment includes conservative substitutions in both a UNC5 binding domain and a DCC/NEO1 binding domain. A NET1 fragment may be a fusion protein that includes a heterologous amino acid sequence. Also provided herein is a composition that includes a NET1 fragment and a pharmaceutically acceptable carrier.

Also provided herein are DCC/NEO1 fragments. In one embodiment, a DCC/NEO1 fragment includes an amino acid sequence having at least 80% identity to amino acids MMPPVGVQASILSHDTIRITWADNSLPKHQKITDSRYYTVRWKTNIPANTKYKNANATT LSYLVTGLKPNTLYEFSVMVTKGRRSSTWSMTAHGATFELVP (SEQ ID NO:18) or MLPPVGVQAVALTHEAVRVSWADNSVPKNQKTSDVRLYTVRWRTSFSASAKYKSEDT TSLSYTATGLKPNTMYEFSVMVTKNRRSSTWSMTAHATTYEAAP (SEQ ID NO:19), wherein the DCC/NEO1 fragment comprises NET1 binding activity. In one embodiment, the DCC/NEO1 fragment includes a NET1 binding domain MVTK(N/G)RRSSTWS (SEQ ID NO:12), wherein the NET1 binding domain includes conservative substitutions in MVTK(N/G)RRSSTWS (SEQ ID NO:12) at positions 1, 2, 3, 4, 5, 6, 7, 8, 9, 10, 11, 12, or a combination thereof. A DCC/NEO1 fragment may be a fusion protein that includes a heterologous amino acid sequence. Also provided herein is a composition that includes a DCC/NEO1 fragment and a pharmaceutically acceptable carrier.

Also provided is antibody. In one embodiment, an antibody is one that specifically binds an epitope located within an amino acid sequence HARRCR (SEQ ID NO:9) or MELYKLS (SEQ ID NO:10). In another embodiment, an antibody is one that specifically binds an epitope located within an amino acid sequence MVTK(N/G)RRSSTWS (SEQ ID NO:12). In another embodiment, an antibody is one that specifically binds to an epitope located within an amino acid sequence of a V-2 fragment CNLHARRCRFNMELYKLSGRKSGGVCLN (SEQ ID NO:16). In another embodiment, an antibody is one that specifically binds to an epitope located within an amino acid sequence of a the V-2 region CNCNLHARRCRFNMELYKLSGRK SGGVCLNCRHNTAGRHCHYCKEGXYRDMGKPITH RKACKAC (SEQ ID NO:17), where the amino acid X is F or Y. An antibody may be a monoclonal antibody or a polyclonal antibody. Also provided are methods for making antibody. including, for instance, administering to an animal a NET1 fragment or a DCC/NEO1 protein.

Also provided are methods for using the fragments and antibody disclosed herein. In one embodiment, a method includes inhibiting binding of a NET1 protein to an UNC5 protein by contacting an UNC5 protein with a NET1 fragment or an antibody, and in another embodiment, a method includes inhibiting binding of a NET1 protein to a DCC/NEO1 protein by contacting a DCC protein with a NET1 fragment or an antibody. In one embodiment, a method includes inhibiting binding of a DCC protein or a NEO1 protein to a NET1 protein by contacting a NET1 protein with a DCC/NEO1 fragment. The contacting may be performed in vitro, in vivo, or ex vivo.

Other methods provided herein include inducing apoptosis of a cell. In one embodiment, a method includes contacting a cell with i) a NET1 fragment and/or ii) an antibody that specifically binds an epitope located within an amino acid sequence HARRCR (SEQ ID NO:9) or MELYKLS (SEQ ID NO:10), SEQ ID NO:16, or SEQ ID NO:17, where the cell expresses a DCC protein, a NEO1 protein, an UNC5 protein, or a combination thereof, on the surface of the cell. In another embodiment, a method includes contacting a cell with i) a DCC/NEO1 fragment and/or ii) an antibody that specifically binds an epitope located within an amino acid sequence MVTK(N/G)RRSSTWS (SEQ ID NO:12), where the cell expresses a DCC protein, a NEO1 protein, or a combination thereof, on the surface of the cell. The cell may be ex vivo or in vivo.

Provided herein is the use of a NET1 fragment or an antibody and a pharmaceutically acceptable carrier, in the manufacture of a medicament for treating a subject having or at risk of having a cancer, and the use of a NET1 fragment or an antibody and a pharmaceutically acceptable carrier, for treating a subject having or at risk of having a cancer. Without intending to be limited by theory, it is believed that a NET1 fragment or an antibody may inhibit binding of UNC5, DCC, NEO1, or a combination thereof, to a NET1 protein. Also provided is the use of a DCC/NEO1 fragment or antibody and a pharmaceutically acceptable carrier, in the manufacture of a medicament for treating a subject having or at risk of having a cancer, and the use of a DCC/NEO1 fragment or an antibody and a pharmaceutically acceptable carrier, for treating a subject having or at risk of having a cancer. Without intending to be limited by theory, it is believed that a DCC/NEO1 fragment or an antibody may inhibit binding of NET1 protein to a NEO1 protein and/or a DCC protein. In one embodiment, the cancer includes a cancer cell selected from colonic carcinoma cell, breast cancer cell, prostate cancer cell, adenocarcinoma cancer cell, neuroblastoma cancer cell, and lung cancer cell. In one embodiment, the subject is a human.

Also provided is a NET1 fragment that includes an alteration of an amino acid corresponding to L359, E385, T415, 1452, or a combination thereof, of a NETT protein, wherein the NET1 fragment will not form a multimer. Further provided is a method for using this type of NET1 fragment, including contacting a cell with the NET1 fragment. The cell may be ex vivo or in vivo. The cell may be a cancer cell, such as a colonic carcinoma cell, a breast cancer cell, a prostate cancer cell, an adenocarcinoma cancer cell, a neuroblastoma cancer cell, or a lung cancer cell.

Also provides is a use of a NET1 fragment that includes an alteration of an amino acid corresponding to L359, E385, T415, 1452, or a combination thereof, of a NET1 protein, and a pharmaceutically acceptable carrier, in the manufacture of a medicament for treating a subject having or at risk of having a cancer, and the use of this type of NET1 fragment and a pharmaceutically acceptable carrier, for treating a subject having or at risk of having a cancer. The cancer may include a cancer cell that is a colonic carcinoma cell, a breast cancer cell, a prostate cancer cell, an adenocarcinoma cancer cell, a neuroblastoma cancer cell, or a lung cancer cell. The subject may be a human.

As used herein, the term “NET1 fragment” refers to a NET1 protein having one or more (e.g., several) amino acids deleted from the amino and/or carboxyl terminus of a mature (processed) NET1 protein.

As used herein, the term “DCC/NEO1 fragment” refers to a fragment of a DCC protein and a fragment of a NEO1 protein, where the fragment has the activity of binding to a NET1 protein and/or fragment, and where the fragment having one or more (e.g., several) amino acids deleted from the amino and/or carboxyl terminus of a mature (processed) DCC protein or a mature (processed) NEO1 protein.

As used herein, the term “UNC5 binding domain” refers to a region of amino acids present on a NET1 protein that plays a role in binding between a NET1 protein and an UNC5 protein.

As used herein, the term “DCC/NEO1 binding domain” refers to a region of amino acids present on a NET1 protein that plays a role in binding between a NET1 protein and a DCC protein, and also plays a role in binding between a NET1 protein and a NEO1 protein.

As used herein, the term “NET1 binding domain” refers to a region of amino acids present on a DCC protein and also present on a NEO1 protein, and plays a role in binding between a DCC protein and a NET1 protein, and also plays a role in binding between a NEO1 protein and a NET1 protein.

As used herein, the term “protein” refers broadly to a polymer of two or more amino acids joined together by peptide bonds. The term “protein” also includes molecules which contain more than one protein joined by disulfide bonds, ionic bonds, or hydrophobic interactions, or complexes of proteins that are joined together, covalently or noncovalently, as multimers (e.g., dimers, tetramers). Thus, the terms peptide, oligopeptide, and polypeptide are all included within the definition of protein and these terms are used interchangeably. It should be understood that these terms do not connote a specific length of a polymer of amino acids, nor are they intended to imply or distinguish whether the protein is produced using recombinant techniques, chemical or enzymatic synthesis, or is naturally occurring.

As used herein, the term “heterologous” refers to an amino acid sequence that is not normally found fused to another amino acid sequence.

As used herein, an “isolated” protein refers to a protein that has been either removed from its natural environment, produced using recombinant techniques, or chemically or enzymatically synthesized.

As used herein, the term “conservative substitution” refers to replacement of one amino acid in a protein with another that maintains certain characteristics of the original amino acid. A conservative substitution for an amino acid in a protein described herein may be selected from other members of the class to which the amino acid belongs. For example, it is well-known in the art of protein biochemistry that an amino acid belonging to a grouping of amino acids having a particular characteristic (such as charge, hydrophobicity, hydrophilicity, and size) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free -OH is maintained; and Gln for Asn to maintain a free —NH2.

As used herein, an antibody that can “specifically bind” a protein is an antibody that interacts only with the epitope of the antigen that induced the synthesis of the antibody, or interacts with a structurally related epitope. An antibody that “specifically binds” to an epitope will, under the appropriate conditions, interact with the epitope even in the presence of a diversity of potential binding targets. As used herein, the term “protein:antibody complex” refers to the complex that results when an antibody specifically binds to a protein.

Conditions that are “suitable” for an event to occur, such as the binding of a ligand, or “suitable” conditions are conditions that do not prevent such events from occurring. Thus, these conditions permit, enhance, facilitate, and/or are conducive to the event.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims. Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIGS. 1A-1D show overall structure of NET1. (FIG. 1A) Ribbon model of NET1 viewed in the “open face” orientation. The domains VI, V-1, V-2, and V-3, are shown. N-linked glycans and disulfide bridges are drawn as sticks, and the calcium ion as the sphere in VI. The asterisk (*) marks amino acid residues 83-85, which could not be traced. (FIG. 1B) Picture of the amino acid interactions mediating the calcium binding. The disulfide bridge between cysteine 119 and 152 is drawn as a stick and the calcium ion is drawn as 1 sphere. (FIG. 1C) Electrostatic potential calculations of the V-domains revealed that V-2 is highly positively charged (IEP: 9.64), a feature only shared with the V-2 subdomain of NET3 (IEP: 9.76). Electrostatic potential is displayed as a gradient from 8 kbT/ec to (+8 kbT/ec. (FIG. 1D) Ab inito envelope (grey) of the NET1 dimer in solution overlaid with the crystal structure of NET1 (darker backbone, top) and its crystal symmetry mate (lighter backbone, bottom). The site 1 interaction uses the α-helix in loop b of V-2 to contact the antiparallel β- sheet of V-3, whereas site 2 is an electrostatic crossover between E385 (V-2 loop d) and T415 (V-3 loop a).

FIGS. 2A-2C show structure determination of NET1. (FIG. 2A) Insight the Ta6Br12 binding site between the N-terminal section of NET1 in the asymmetric unit (left) and a crystal symmetry mate (right). The Difference Fourier map is displayed at 4 Sigma contour level. In the refined cluster Ta and Br are depicted as spheres. Both binding partners form a hooklike arrangement to fix the cluster in a tight pocket. (FIG. 2B) Difference Fourier map using the 5-SAD anomalous signal measured at 1.9 Å wavelength. Individual Fourier peaks (4.0 Sigma contour level) for all 17 disulfide bridges are shown with the refined cysteine positions shown as sticks. The anomalous signal allowed for the interpretation of the calcium signal (Fourier peak).

(FIG. 2C) Representative electron density map in the refined structure. Regions of all three Nlinked glycan moieties are shown. Both panels show Sigma-A weighted 2Fo-Fc maps at 2.6 A resolution (1.5 Sigma contour level).

FIG. 3 shows secondary structure diagram of NET1. Disulfide connectivity in domains VI and V are indicated by dotted lines, visible N-linked glycans by 4-point stars with the Asn residues N95, N116 and N131 highlighted. The structural calcium at the C-terminal end of the helix between rS2 and Si is indicated as a sphere. β-strands are depicted as arrows and the α-helices as cylinders, each labeled with the first and last residue. The cysteine residues 1-3, 2-4, 5-6, and 7-8 in subdomains V-1 to V-3 are bridged together forming the loop segments a-d accordingly.

FIGS. 4A-4B show comparision of different N-terminal domains from Netrin G proteins and laminin short arms. (FIG. 4A) Individual N-terminal domains of NG1 (pdb-code: 3TBD) (Brasch et al., J Mol Biol 414, 723 (2011)), NG2 (pdb-code:3ZYI) (Seiradake et al., EMBO J 30, 4479 (2011)), laminin-α5 (pdb-code:2Y38) (Hussain et al., EMBO Rep 12, 276 (2011)), laminin-β1 (pdb-code:4AQS) and laminin-γ1 (pdb-code:4AQT) (Carafoli et al., PLoS One 7, e42473 (2012)) are shown in ribbon diagram representation. Disulfide bridges 1 to 4 between cysteine residues 1-2, 3-6, 4-5 and 7-8 are highlighted as spheres and marked in brackets. Phosphate ions in NG1 and laminin-α5 are shown as spheres shaded according atom type. N-linked sugar moieties are represented in stick mode. The front face is shown in the upper row and the dorsal face in the lower row. (FIG. 4B) The VI domain of mouse NET1 (mNET1; NP_032770; SEQ ID NO:20), and the LN domains of mouse laminin-β1 (mLamβ1; NP_032508; SEQ ID NO:21), mouse laminin-α5 (mLamα5; NP_001074640; SEQ ID NO:22), and mouse laminin-γ1 (mLamγ1; NP_034813; SEQ ID NO: 23) were aligned using MULTALIN (Corpet, Nucleic Acids Res 16, 10881 (1988)) and formatted with ESPRIPT (Gouet et al., Bioinformatics 15, 305 (1999)). The organization of the disulfide bridges are indicated by lines marking the cysteine residues on either side. Note, that the disulfide bridge formed between 7 and 8 is unique for NET1. Putative glycosylation sites are underlined.

FIGS. 5A-5B show biophysical characterization of NET1. (FIG. 5A) Elution profiles obtained for NET1 by size exclusion chromatography (Superdex™ 200 10/300) in 0.05 M Tris/Tris-HCl, pH 7.5, 0.2 M NaCl (line with three peaks) and in 0.05 M Tris/Tris-HCl, pH 7.5, 1.0 M NaCl, 0.15 M glycine (line with one peak). The two peaks in 0.05 M Tris/Tris-HCl, pH 7.5, 0.2 M NaCl represent a predominantly monomeric form (15 ml peak, Rh 4 nm) and a multimeric form (11 ml peak, Rh 8 nm) indicating that at low salt concentrations, NET1 is self-associating. In 0.05 M Tris/Tris-HCl, pH 7.5, 1.0 M NaCl, 0.15 M glycine a single elution peak at 14 ml was obtained, corresponding to a dimer with Rh 4.7 nm (see also Table 3). The inset shows concentration dependence of the Stokes radius (Rh) of NET1, as determined by dynamic light scattering in 0.05 M Tris/Tris-HCl, pH 7.5 supplemented with either 0.15 M NaCl (upper line) or 1.0 M NaCl, 0.15M glycine (lower line). In the presence of 1.0 M NaCl, 0.15 M glycine an Rh of 5.24±0.24 nm was obtained that did not change significantly with increasing protein concentration. This value is corresponding to a NET1 dimer in solution. At a lower salt content of 0.15 M NaCl we observed an increase of Rh from 7.85 ±0.03 (at 1.36 mg/ml) to 8.9±0.3 nm (at 3.4 mg/ml) indicating self-association behavior of NET1. (FIG. 5B) Raw SAXS data for NET1 in 0.05 M Tris/Tris-HCl, pH 7.5, 1.0 M NaCl, 0.15M glycine are shown as dots with the fitted calculated model as black line. The P(r) function is shown as inset.

FIGS. 6A-6E show two distinct binding epitopes in V-2 mediate dependence receptor binding. (FIG. 6A) The cartoon represents different NET1 chimeras. (FIG. 6B) Solid-phase binding assays with NET1 and different chimeras demonstrate the binding activities to immobilized NEO1 (left) and UNC5h2 (right). The curves are plotted according the fraction bound. The inset shows the binding behaviour of different mutations normalized to wild type NET1. (FIG. 6C) X-ray structure of the NEO1-binding motif at the helical segment 354MELYKLS360 and the UNC5h2-binding motif at R348-R349, respectively. (FIG. 6D) HEK293 cell binding studies to different coated substrates. NET1 is shown as circles, NET1 R348A-R349A double mutant is shown as triangles, and the Δ354MELYKLS360 version is shown as diamonds. (FIG. 6E) Microscopic pictures of wells from a cell adhesion assay with different substrates. The images at the top taken at 4× amplification and stained with crystal violet give an overview of the adhered cells. The bottom pictures taken at 10× amplification show cells spreading of the cells on the respective substrates.

FIGS. 7A-7E show solid-phase binding studies of NET1 and different chimeras to decipher binding epitopes for DCC, NEO1 and UNC5h2. (FIG. 7A) Cartoons symbolizing the domain swaps between NET1 and laminin-γ1. (FIG. 7B-7E) In solid binding assays the receptor ectodomains of DCC, NEO1, and UNC5h2 were coated and incubated with serial diluted ligands NET1 and the different chimeras. All experiments were performed three times in triplicate. The graphs clearly prove that all NET1 chimera containing the V-2 subdomain (Chimera 2, 3 and 5) can bind to the ectodomains of all three receptors. In contrast, Chimera 1 and 4 lacking subdomain V-2 do not interact with the full-length ectodomain of DCC, NEO1, and UNC5h2.

FIGS. 8A-8C show Surface Plasmon Resonance studies of NET1 and different chimera to decipher binding epitopes for DCC, NEO1 and UNC5. SPR analysis was performed as an independent second method to define the binding region within NET1 to its respective receptors. Receptors NEO1 (FIG. 8A), DCC (FIG. 8B) and UNC5h2 (FIG. 8C) were coupled to a CMS chip. The analyte NET1 as well as different chimera proteins were passed in a serial dilution from 30 nM, 100 nM and 300 nM over the coupled chip. SPR graphs confirm the observations from the solid-phase binding assay.

FIGS. 9A-9B show sequence alignment and structural comparison of different V subdomains of NET1, NET3, NET4 with the LE domains of laminin-α5, -β1-and -γ1. (FIG. 9A) The V2 domain of mNET1 (NP_032770; SEQ IS NO:24), mNET3 (NP_035077; SEQ ID NO:25), and mNET4 (NP_067295; SEQ ID NO:26) as well as the second LE domain of mLamβ1 (NP_032508; SEQ ID NO:27) mLamα5 (NP_001074640; SEQ ID NO:28), and mLamγ1 (NP_034813; SEQ ID NO:29) were aligned as described above (see FIG. 16). The eight cysteine residues in each subdomain V are disulfide linked in the order 1-3, 2-4, 5-6, and 7-8 creating the four loop segments a-d, respectively. Whereas NET1 -and 3 are binding the dependence receptors NEO1/DCC and UNC5, NET4 and all laminins do not interact with dependence receptors. The residues studied in the mutation analysis are marked by * and numbers. (FIG. 9B) Insight view of the loop a and b segments. The V-2 subdomain of NET4 was homology modeled based on the NET1 crystal structure.

FIGS. 10A-10B show determination of cell binding to NET1 and identification of the respective binding domain within NET1. (FIG. 10A) Graph displays a cell adhesion assay in which NET1 as well as different chimera proteins are used as substrate for HEK293 cells. The color code is according to FIG. 7. The adhesion assays show that chimera comprising the NET1 subdomain V-2 (Chimera 2, 3, and 5) mediate cell adhesion of HEK293 cells, whereas chimeras missing the subdomain V-2 (Chimera 1 and 4) do not. (FIG. 10B) The cell binding to NET1 is most likely integrin independent, since HEK293 binding to NET1 is not inhibited by the addition of EDTA and not significantly changed by Mn2+ and Mg2+ addition.

FIGS. 11A-11B show transcript and protein level detection of NET1 receptors in HEK293 cells. (FIG. 11A) RT-PCR analysis shows that HEK293 cells express NEO1 and UNC5h2, but not DCC. Further determination of NET1 receptor mRNA levels within different tissues, such as fetal and adult brain, kidney and lung reveals similar amounts of NEO1/DCC mRNA for all tissues. UNC5h2 mRNA is present within the kidney, lung, and fetal brain but not within adult brain. (FIG. 11B) Comparison of the protein level in HEK293 cells via Western Blot analysis confirms the expression analysis. In addition, the human melanoma cell line A431 displays the same NET1 receptor expression pattern as observed in the HEK293 cell line.

FIGS. 12A-12D show Dependence Receptors interacting with NET1. (FIG. 12A) SAXS model of the full-length ectodomain of NEO1. Rigid body fitting was possible because all individual domains were determined crystallographically (see also FIGS. 13 and 14). The F-G loop segment of the 946MVTKNRRSSTW956 (SEQ ID NO:8) peptide is shown as atomic model. (FIG. 12B) Cartoon of NEO1 and Solid-Phase analysis to map the NET1 binding epitope. (FIG. 12C) The NEO1-NET1 complex in the absence and presence (480 μg/ml) of heparin shows identical binding. (FIG. 12D) Cartoon of UNC5h2 and the solid-phase binding assay with NET1 that indicates that the C-terminal TSP tandem is not involved in binding. The inset demonstrates that Ig1 mediates binding to NET1, whereas Ig2 does not.

FIGS. 13A-13C show biophysical characterization of the full-length ectodomain of NEO1, NEO1 ΔFN[5-6] and the NEO1-NET1 complex. (FIG. 13A) DLS profile for the full-length ectodomain of NEO1, NEO1 ΔFN[5-6] and the NEO1-NET1 complex indicating that the NEO1-NET1 complex is larger than NEO1 itself, whereas the NEO1 ΔFN[5-6] truncated version is smaller than the fulllength ectodomain. (FIG. 13B) Raw Scattering data for NEO1, NEO1 ΔFN[5-6] and the NEO1-NET1 complex. (FIG. 13C) Sequence alignment of the FN[5] domains of murine NEO1 (AAH54540; SEQ ID NO:30) and murine DCC (NP_031857; SEQ ID NO:31) using bl2seq (NCBI) and formatted with ESPRIPT. Conserved amino acids, which were mutated, are indicated by asterisks.

FIGS. 14A-14D show solution X-ray scattering studies of NEO1 (full-length ectodomain) and NEO1 AFN[5-6]. (FIG. 14A) Raw SAXS data for NEO1 are shown as dots with the data calculated from individual models representing the goodness of fit (χ-values) as colored lines. The P(r) function that yield the radius of gyration (Rg) and maximal particle dimension (Dmax) is shown in the inset. (FIG. 14B) To verify the domain arrangement we performed a proof-of-principle experiment and compared the full-length structure with a truncated version, where the C-terminal domains FN [5-6] were truncated. Raw SAXS data for NEO1 ΔFN[5-6] are shown as diamonds with the fitted calculated model as colored line. The P(r) function is shown in the inset. (C+D) Rigid body models for NEO1 (FIG. 14C) and NEO1 ΔFN[5-6] (FIG. 14D). The models were built using high-resolution crystal structures of Ig1-4 (residues 24-404; pdb:3LAF), FN[1] (residues 431-529;pdb:1X5F), FN[2-3] (residues 534-718; pdb:3LPW), FN[4] (residues 728-825; pdb:1X5I, and FN[5-6] (residues 831-1030; pdb:3P4L) (Yang et al., J Struct Biol 174, 239 (2010)).

FIG. 15 shows heparin binding properties of NEO1. The doubly kinked structure of the FN[3-5] tandem is shown at the bottom panel with the bound sucrose-octasulphate (SOS) molecule projected. The position of SOS was taken from RCSBID 4BQC (Bell et al., Science 341, 77 (2013)). The electrostatic potential is depicted as a gradient ranging from −8 kbT/ec to +8 kbT/ec. The top panel shows a close-up view of the crevice formed between F-G and B-C loop elements at FN[5] (left) and FN[3] (right). Remarkably, the neck at both ends is flanked by various basic charged residues that were shown to be involved in heparin-binding (Bennett et al., J Biol Chem 272, 26940 (1997); Geisbrecht et al., J Biol Chem 278, 32561 (2003); Kruger et al., J Neurosci 24, 10826 (2004)). It is tempting to speculate that heparin binding might be involved in proper hinge formation of the extended FN tandem structure.

FIGS. 16A-16G show molecular mechanisms of NET1-mediated dependence receptor binding. (FIG. 16A) NEO1-NET1 complex reconstitution by SEC. Monomeric NET1 (Rh 4.0 nm) and NEO1 (Rh 7.0 nm) were separately purified. The purified components were mixed in a 1:1 molecular ratio, concentrated and reapplied to the SEC column (curve with first peak). An elution peak that corresponds to a larger hydrodynamic volume (Rh 8.4 nm) indicates complex formation. This peak was used for SAXS studies. The hydrodynamic scale at the top was derived from calibration runs using marker proteins (see FIG. 17). (FIG. 16B) To obtain the SAXS shape of the NEO1-NET1 complex, we fitted SAXS data for NEO1 (lower) and NEO1-NET1 (upper trace) simultaneously. Data back calculated from individual models of NEO1 as well as the complex are plotted in colored lines, which provides the goodness of fit parameter (x value). The inset figure presents the P(r) function for the NEO1-NET1 complex that provides Rg and Dmax parameters (see also Table 3). (FIG. 16C) Rigid body model of the NEO1-NET1 complex obtained from the scattering data is shown in surface presentation. The spot in region V marks the UNC5h2-binding motif. (FIG. 16D) Structural comparison of nidogen-laminin with NEO1-NET1. (FIG. 16E) Solid-phase binding assay indicates a constant binding ratio of NET1 (125 nM) to NEO1 if increasing UNC5h2 quantities are added. (FIG. 16F) Model of NET1 signaling. The monomeric form of NET1 can bind simultaneously the ectodomains of both types of dependence receptors. In addition, the dimeric entity of NET1 allows for the multimerization of either UNC5 or NEO1/DCC in a 2:2 molar ratio. (FIG. 16G) Neuronal branching points (arrows) of sensory neurons cultured on printed substrates. Bands of laminin-111 substrates were crossed with stripes of NET1, the NET1 R348A-R349A double mutant or NET1 Δ354MELYKLS360 truncation. Scale bars represent 25 μm and white bars indicate the direction of laminin-111 bands. Shaded line depicts NET1 and mutant stripes.

FIG. 17 shows hydrodynamic calibration curve through marker proteins. The hydrodynamic radii of the eluted NET1, NEO1, and the NEO1-NET1 complex (FIG. 16) were determined by means of a calibration curve obtained from aprotinin from bovine lung (Rh 1.35 nm), cytochrome c from equine heart (Rh 1.77 nm), carbonic anhydrase from bovine erythrocytes (Rh 2.35 nm), ovalbumin from chicken egg (Rh 2.98 nm), conalbumin from chicken egg white (Rh 3.64 nm), aldolase from rabbit muscle (Rh 4.77 nm), catalase from bovine liver (Rh 5.22 nm), ferritin from horse spleen (Rh 6.71 nm) and thyroglobulin from bovine thyroid (Rh 8.58 nm). The void volume of the column was determined to be 8.54 ml using a soluble form of prohibitin-1, which forms a giant supramolecular complex (courtesy of Dr. G. P. Padilla-Meier).

FIG. 18 shows rigid body modeling of the NEO1-NET1 complex. Multiple rigid-body models were calculated from SAXS data for the complex between NEO1 and NET1. The NET1 molecule is situated at the top of each model (horizontal orientation) while NEO1 is located below. The horseshoe structure of NEO1 is found at the bottom of each model. Both x values for each model represent the goodness of fitted data with NEO1 and the NEO1-NET1 complex, respectively. The NSD value for each dataset is presented in Table 3.

FIGS. 19A-19B show NEO1-binding motif of NET1. (FIG. 19A) Overview of the NEO1-NET1 complex. NET1 is illustrated as antiparallel dimer in surface presentation for the V-2 (upper left shaded region) and V-3 (lower right shaded region) subdomains. The individual FN-III domains are labeled accordingly and a projected SOS molecule is drawn as spheres. The relative orientation of individual NET1 molecules does allow for the concurrent binding of two NEO1 molecules and the formation of a NEO1-NET1 complex with a 2:2 molar ratio. (FIG. 19B) In-depth view of the NEO1-NET1 heterocomplex. Individual amino acid residues, which were identified through site-directed mutagenesis are highlighted.

FIG. 20A shows neuronal branching. Sensory neurons were cultured on printed substrates where bands of recombinant laminin-111 were crossed with stripes of a test protein. Neurite outgrowth was predominantly observed on the recombinant laminin-111 substrate. Initiated junctions were scored by counting neurite branch points at the junctions between recombinant laminin-111 and the test protein (marked by arrows). There were significantly more branch points per unit length observed when the test substrate was recombinant laminin-111 or NET1 compared to chimera 1 (Student's t test; p<0.001). Representative images of cells cultured on different substrates. Scale bars are 25 μm. White bars indicate the direction of recombinant laminin-111 bands, while shaded bars indicate NET1 stripes and chimera 1 bands in NET1 and chimera 1 substrates, respectively. All data were obtained from triplicate experiments.

FIGS. 21-1 through 21-6 show amino acid sequences of examples of Netrin-1 proteins from Mus musculus and from Homo sapiens, Neogenin (NEO1) proteins from Mus musculus and from Homo sapiens, DCC proteins from Mus musculus and from Homo sapiens, and an Unc5 protein from Mus musculus.

### DETAILED DESCRIPTION OF ILLUSTRATIVE EMBODIMENTS

### Proteins

Provided herein are fragments of a Netrin-1 (NET1) protein that bind to a dependence receptor UNC5, to DCC, to the DCC paralogue NEO1, or a combination thereof. In some aspects described herein both DCC and NEO1 are referenced together as DCC/NEO1, thus DCC/NEO1 refers to a DCC protein and a NEO1 protein, and not to a chimera of DCC and NEO1. A mature NET1 protein is a cysteine-rich glycoprotein that activates cell surface receptors to initiate multiple downstream signal transduction pathways. A NET1 protein may be from a vertebrate, such as an avian species or a mammal. Examples of mammals include, for instance, mouse and human. NET1 proteins are highly conserved among species, and a NET1 protein from any species may be used as the source of a NET1 fragment described herein. Examples of NET1 proteins are depicted at SEQ ID NO:1 (NCBI number AAC52971, Mus musculus) and SEQ ID NO:2 (NCBI number AAD09221, Homo sapiens). In one embodiment a NET1 fragment is a monomer. In one embodiment a NET1 fragment is a multimer, either a homomultimer or a heteromultimer. A multimer may be a dimer, a trimer, a tetramer, etc. In one embodiment a NET1 fragment is a dimer, either a homodimer or a heterodimer.

In one embodiment, a NET fragment binds both an UNC5 protein and a DCC/NEO1 protein. In one embodiment a NET1 fragment binds to an UNC5 protein and does not bind a DCC/NEO1 protein, e.g., does not bind a DCC protein, does not bind a NEO1 protein, or does not bind either protein. In one embodiment a NET1 fragment binds to a DCC/NEOlprotein and does not bind an UNC5 protein.

In those embodiments where a NET1 fragment binds an UNC5 protein, the NET1 fragment includes UNC5 binding domain HARRCR (e.g., amino acids 344-349 of SEQ ID NO:1, amino acids 345-350 of SEQ ID NO:2). The HARRCR (SEQ ID NO:9) may include between 1 and 5 conservative substitutions at positions 1, 2, 3, 4, 5, 6, or a combination thereof, and maintain binding to an UNC5 protein. In one embodiment, the conservative substitution of the arginine at residue 3, 4, and/or 6 of the UNC5 binding domain HARRCR (SEQ ID NO:9) is selected from a histidine or a lysine. In one embodiment, the cysteine at residue 5 is a serine. In one embodiment, the conservative substitution in HARRCR (SEQ ID NO:9) is at positions 1, 2, or a combination thereof.

In those embodiments where a NET1 fragment has reduced binding to an UNC5 protein (e.g., a NET1 fragment has reduced binding to UNC5 but does bind DCC or NEO1), the NET1 fragment includes an alteration of the amino acid sequence of HARRCR (SEQ ID NO:9) that decreases the binding of the altered NET1 fragment to an UNC5 protein. In one embodiment, the alteration is a non-conservative substitution at positions 1, 2, 3, 4, 5, 6, or a combination thereof. In one embodiment the alteration is a deletion of residue 1, 2, 3, 4, 5, 6, or a combination thereof. In those embodiments where a NET1 fragment binds a DCC/NEO1 protein, the NET1 fragment includes DCC/NEO1 binding domain MELYKLS (amino acids 353-359 of SEQ ID NO:1, amino acids 354-360 of SEQ ID NO:2). The MELYKLS (SEQ ID NO:10) may include between 1 and 7 conservative substitutions at positions 1, 2, 3, 4, 5, 6, 7, or a combination thereof, and maintain binding to a DCC/NEO1 protein. In one embodiment, the conservative substitution of the glutamic acid at residue 2 of the DCC/NEO1 binding domain is an aspartic acid. In one embodiment, the conservative substitution of the lysine at residue 5 of the DCC/NEO1 binding domain is an arginine or a histidine.

In those embodiments where a NET1 fragment has reduced binding to a DCC/NE01 protein (e.g., a NET1 fragment does not bind DCC or NEO1 but does bind UNC5), the NET1 fragment includes an alteration of the amino acid sequence of MELYKLS (SEQ ID NO:10) that decreases the binding of the altered NET1 fragment to a DCC/NEO1 protein. In one embodiment, the alteration is a non-conservative substitution at positions 1, 2, 3, 4, 5, 6, 7, or a combination thereof. In one embodiment the alteration is a deletion of residue 1, 2, 3, 4, 5, 6, 7, or a combination thereof.

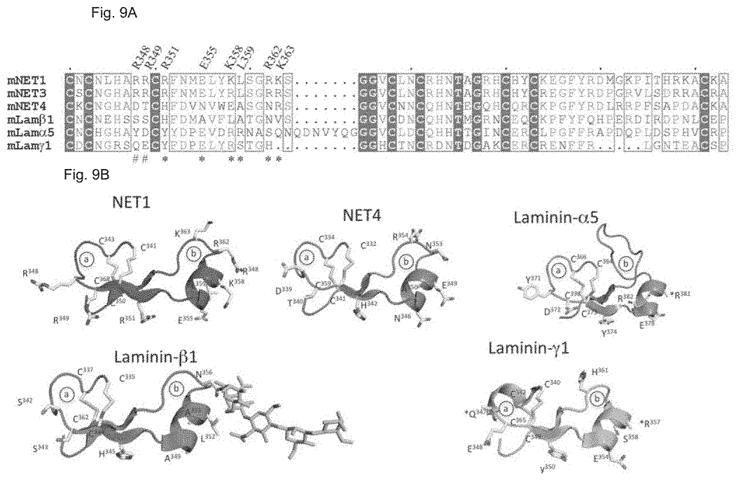

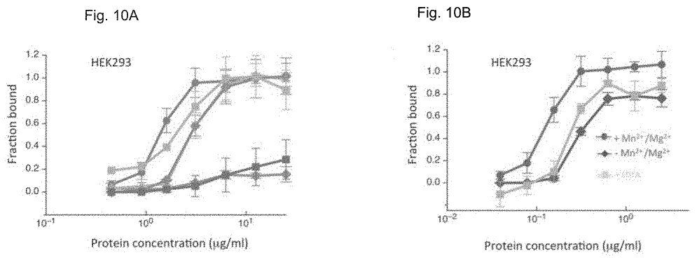

In one embodiment, a NET1 fragment includes CNLHARRCRFNMELYKLSGRKSGGVCLN (SEQ ID NO:16). This NET1 fragment corresponds to a portion of the V-2 region of a mature NET1 protein, and thus is also referred to herein as a V-2 fragment, and is depicted at FIG. 9B. Amino acids corresponding to the UNC5 binding domain and amino acids corresponding to the DCC/NEO1 binding domain may be present in a V-2 fragment.

In one embodiment, a NET1 fragment includes CNCNLHARRCRFNMELYKLSGRK SGGVCLNCRHNTAGRHCHYCKEGXYRDMGKPITH RKACKAC (SEQ ID NO:17), where the amino acid X is F or Y. This NET1 fragment corresponds to the V-2 region of a mature NET1 protein, and thus is also referred to herein as a V-2 region. Amino acids corresponding to the UNC5 binding domain and amino acids corresponding to the DCC/NEO1 binding domain may be present in a V-2 fragment.

In one embodiment, a NET1 fragment consists of HARRCR (SEQ ID NO:9), MELYKLS (SEQ ID NO:10), the amino acids of a V-2 fragment, the amino acids of a V-2 region, or a protein having structural similarity to the amino acids of a V-2 fragment or a V-2 region, provided the NET1 fragment binds UNC5, DCC/NEO1, or a combination thereof. In one embodiment, a NET1 fragment further includes additional amino acids at the amino-terminal end, the carboxy-terminal end, or both ends. The number of amino acids at the amino-terminal end may be at least 5, at least 10, at least 50, or at least 100, and may be no greater than 10, no greater than 50, or no greater than 150. The number of amino acids at the carboxy-terminal end may be at least 5, at least 10, at least 50, or at least 100, and may be no greater than 10, no greater than 50, or no greater than 150. In one embodiment the additional amino acids are from a mature NET1 protein. The additional amino acids may be part or all of a V-1 region, part or all of a VI region, part or all of a V-3 region, and/or part or all of a C′ region.

In one embodiment, the additional amino acids are heterologous amino acids. The skilled person can easily determine whether an amino acid sequence located at an end of a NET1 fragment is heterologous by comparison of the amino acid sequence to the corresponding region of a mature NET1 protein. A NET1 fragment that includes heterologous amino acids may be referred to as a fusion protein.

Further provided are fragments of a neogenin (NEO1) protein that bind a NET1 protein. A NEO1 protein may be from a vertebrate, such as an avian species or a mammal. Examples of mammals include, for instance, mouse and human. NEO1 proteins are highly conserved among species, and a NEO1 protein from any species may be used as the source of a NEO1 fragment described herein. Examples of NEO1 proteins are depicted at SEQ ID NO:3 (NCBI number AAH54540, Mus musculus) and SEQ ID NO:4 (NCBI number AAI43272, Homo sapiens).

Also provided are fragments of a Delayed in Colon Cancer (DCC) protein that bind a NET1 protein. A DCC protein may be from a vertebrate, such as an avian species or a mammal. Examples of mammals include, for instance, mouse and human. DCC proteins are highly conserved among species, and a DCC protein from any species may be used as the source of a DCC fragment described herein. Examples of DCC proteins are depicted at SEQ ID NO:5 (NCBI number NP 031857, Mus musculus) and SEQ ID NO:6 (NCBI number CAA53735, Homo sapiens).

Both a NEO1 fragment and a DCC fragment each independently bind a NET1 protein at the same location on a NET1 protein (i.e., at the DCC/NEO1 binding site located on a NET1 protein as described herein). Since they both bind a NET1 protein they are referred to herein together as a DCC/NEO1 fragment. A DCC/NEO1 fragment also binds a NET1 fragment that includes a DCC/NEO1 binding domain. The skilled person will recognize that a DCC/NEO1 fragment will also bind NET1 proteins that are described in the art, provided the NET1 protein includes a DCC/NEO1 binding domain.

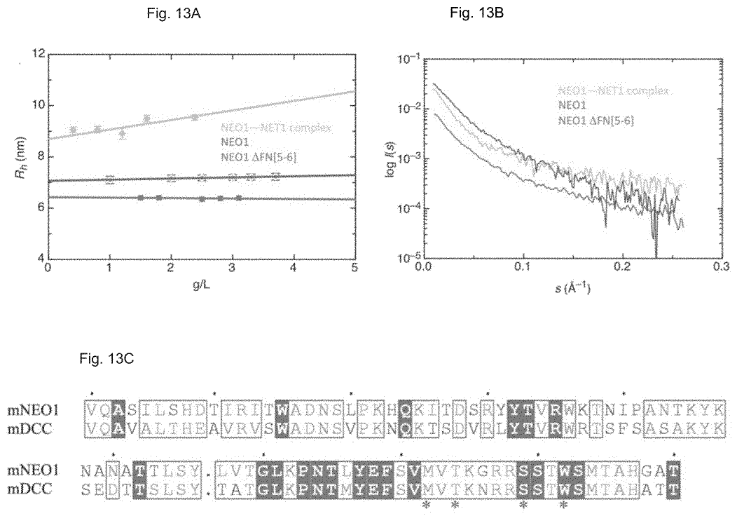

A DCC/NEO1 fragment includes a NET1 binding domain MVTK(N/G)RRSSTWS (SEQ ID NO:12), where the amino acid at position 5 is N or G. The MVTK(N/G)RRSSTWS may include between 1 and 12 conservative substitutions at any position. In one embodiment, the M, the T at position 3, the S at position 8, the W, or a combination thereof, are not substituted (see FIG. 13C). In one embodiment, 1, 2, 3, or 4 amino acids of the sequence K(N/G)RR (SEQ ID NO:11) are substituted with a non-conservative substitution. A NET1 binding domain is expected to form an antiparallel beta-sheet, and any substitution that disrupts that structure is expected to decrease the ability of a NET1 binding domain to bind to NET1. Using the information contained herein, the skilled person can predict which substitutions are likely to alter the structure of a NET1 binding domain.

In one embodiment, a DCC/NEO1 fragment includes MVTKNRRSSTW (e.g., amino acids 946-955 of SEQ ID NO:3), and in another embodiment includes MVTKNRRSSTWS (amino acids 921-932 of SEQ ID NO:5). This fragment is located in fibronectin type III (FN) domain 5 of a NEO1 protein. In one embodiment a DCC/NEO1 fragment consists of the amino acids of a NET1 binding domain MVTK(N/G)RRSSTWS (SEQ ID NO:12) or a DCC/NEO1 fragment having structural similarity to the NET1 binding domain provided the DCC/NEO1 fragment binds NET1.

In one embodiment a DCC/NEO1 fragment includes MMPPVGVQASILSHDTIRITWADNSLPKHQKITDSRYYTVRWKTNIPANTKYKNANATT LSYLVTGLKPNTLYEFSVMVTKGRRSSTWSMTAHGATFELVP, which is SEQ ID NO:18 (e.g., amino acids 868-968 of SEQ ID NO:3). This fragment is the fibronectin type III (FN) domain 5 of a NEO1 protein. In one embodiment a DCC/NEO1 fragment includes MLPPVGVQAVALTHEAVRVSWADNSVPKNQKTSDVRLYTVRWRTSFSASAKYKSEDT TSLSYTATGLKPNTMYEFSVMVTKNRRSSTWSMTAHATTYEAAP, which is SEQ ID NO:19 (e.g., amino acids 843-943 of SEQ ID NO:5). This fragment is the fibronectin type III (FN) domain 5 of a DCC protein. In one embodiment a DCC/NEO1 fragment consists of the FN domain 5 of a NEO1 protein or a DCC protein or a protein having structural similarity to a FN domain 5.

In one embodiment a DCC/NEO1 fragment includes MVTKNRRSSTW (e.g., amino acids 946-956 of SEQ ID NO:3) and includes other amino acids. In one embodiment a DCC/NEO1 fragment further includes additional amino acids at the amino-terminal end, the carboxy-terminal end, or both ends. The number of amino acids at the amino-terminal end may be at least 5, at least 10, at least 50, or at least 100, and may be no greater than 10, no greater than 50, or no greater than 150. The number of amino acids at the carboxy-terminal end may be at least 5, at least 10, at least 50, or at least 100, and may be greater than 10, no greater than 50, or no greater than 150.

In one embodiment, the additional amino acids are from a NEO1 protein. For instance, at the amino-terminal end the additional amino acids are selected from amino acids upstream of MVTKNRRSSTW, e.g., amino acids 868-945 of SEQ ID NO:3 (or a series of amino acids having structural similarity thereto), and at the carboxy-terminal end the additional amino acids are selected from amino acids downstream of MVTKNRRSSTW, e.g., amino acids 957-968 of SEQ ID NO:3 (or a series of amino acids having structural similarity thereto). In one embodiment the additional amino acids are from a DCC protein. For instance, at the amino-terminal end the additional amino acids are selected from amino acids upstream of MVTKNRRSSTWS, e.g., amino acids 843-920 of SEQ ID NO:5 (or a series of amino acids having structural similarity thereto), and at the carboxy-terminal end the additional amino acids are selected from amino acids downstream of MVTKNRRSSTWS, e.g., amino acids 933-943 of SEQ ID NO:5 (or a series of amino acids having structural similarity thereto).

In one embodiment, the additional amino acids are heterologous amino acids. The skilled person can easily determine whether an amino acid sequence located at an end of a DCC/NEO1 fragment is heterologous by comparison of the amino acid sequence to the corresponding region of a mature NEO1 protein or a mature DCC protein. A DCC/NEO1 fragment that includes heterologous amino acids may be referred to as a fusion protein.

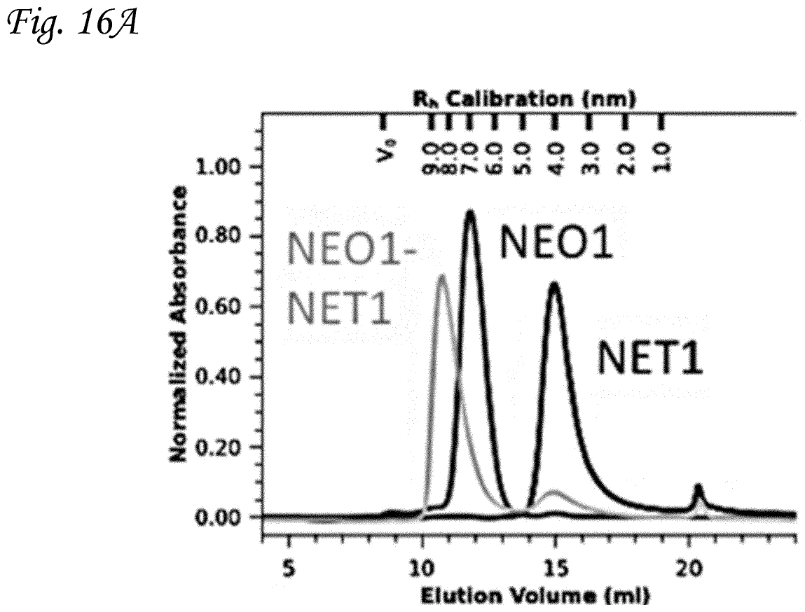

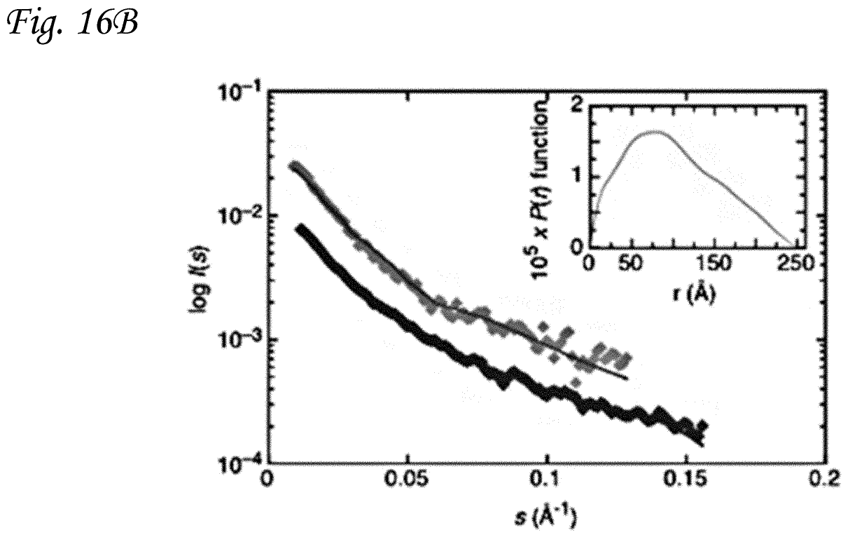

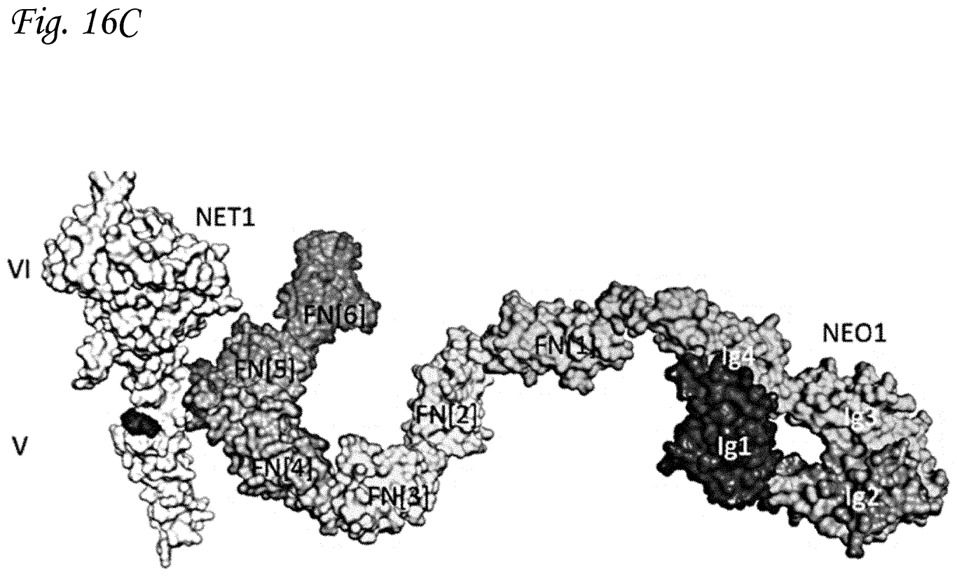

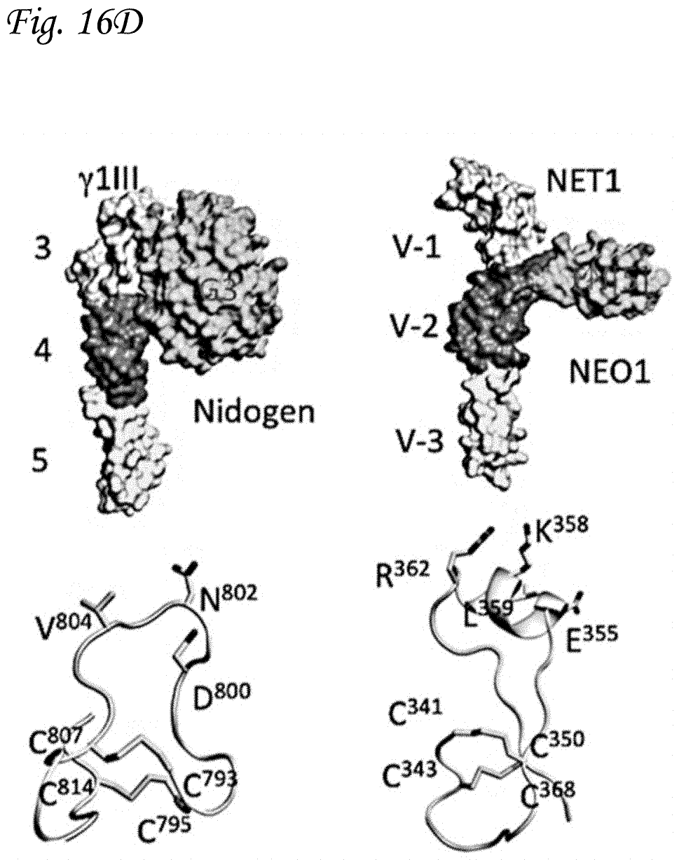

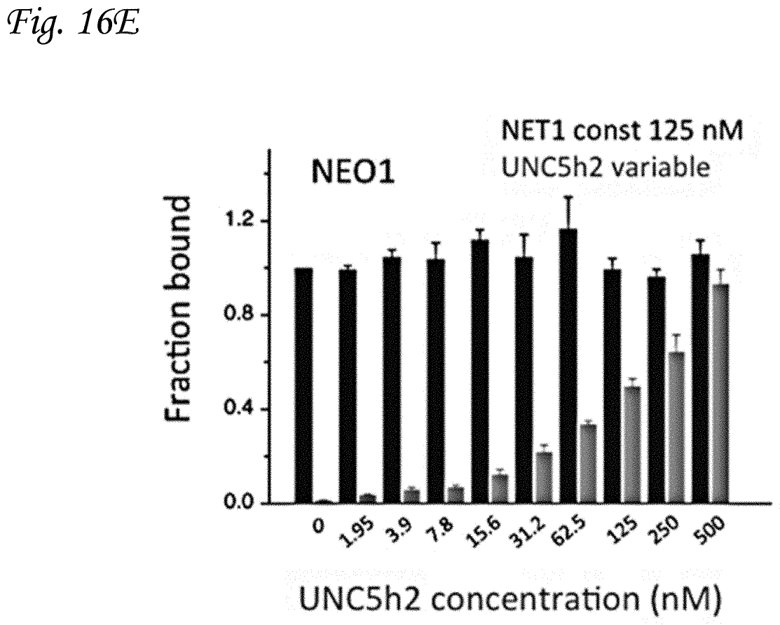

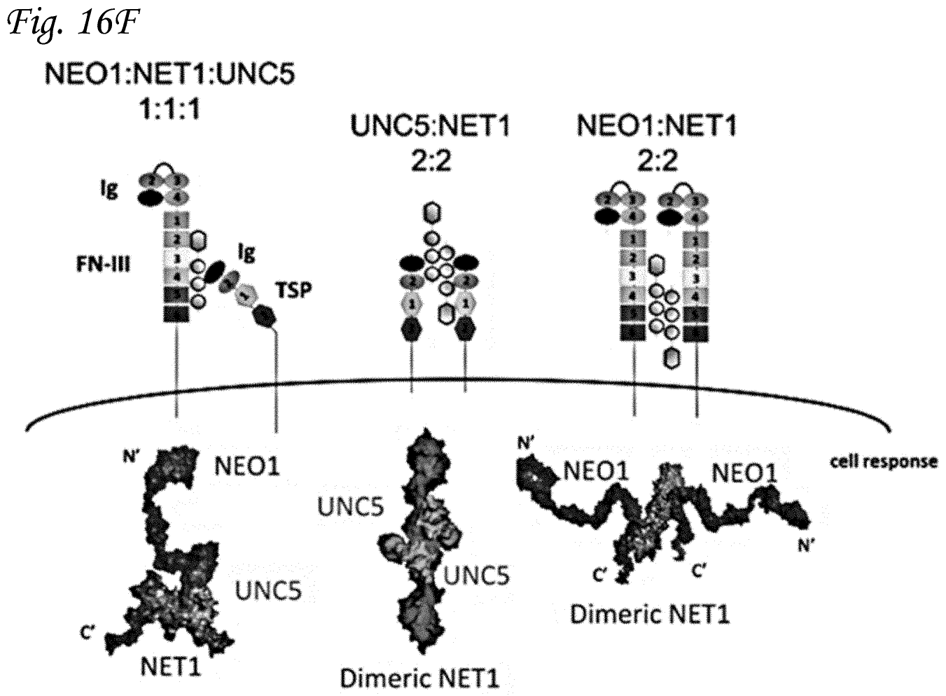

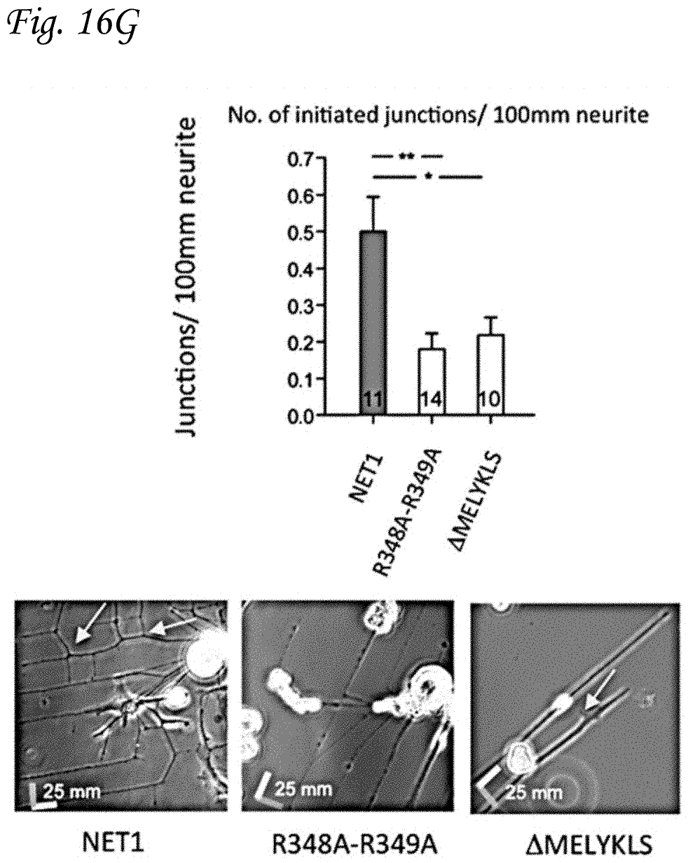

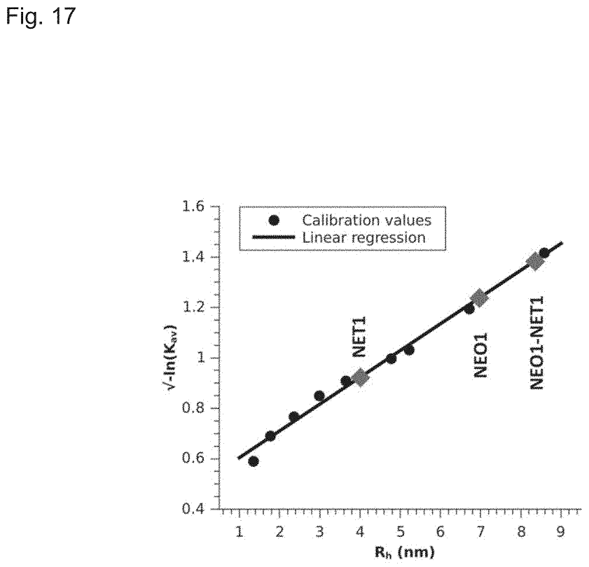

Net1 proteins have the ability to form homomultimers, and it is believed that Net1 multimers, e.g., homodimers, play an role in the biological activities of Net1. For instance, it has been predicted that Net1 dimers mediate the dimerization of dependence receptors including UNC5, DCC, and NEO1, and the dimerization of these receptors affects the biology of a cell. Such dimerization is depicted in FIG. 16F. Also provided herein are Net1 proteins and Net1 fragments having a decreased ability to form a multimer, e.g., a dimer, with another Net1 protein and/or a Net1 fragment.

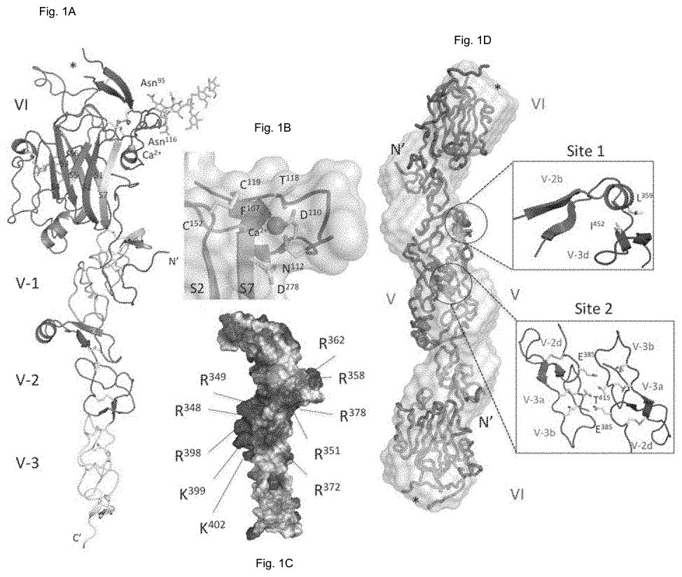

Based on the structural data described herein, it is predicted that certain amino acids of Net1 are involved in formation of a Net1 homodimer (see Site 1 and Site 2 of FIG. 1D). The amino acids include L359, E385, T415, and/or 1452 of SEQ ID NO:2. A person of ordinary skill in the art recognizes that the precise location of these residues in a Net1 protein can vary between members of different species, and between members of the same species, due to variation that naturally exists. The locations of the residues in a Net1 protein are approximate, and can vary by 1, 2, 3, 4, or about 5. For instance, the L359, E385, T415, 1452 amino acids of the Net1 protein shown at SEQ ID NO:2 are present at residues 358, 384, 414, and 451, respectively, in the Net1 protein shown at SEQ ID NO:1. The location of the L359 residue in a Net1 protein can be determined by locating the amino acid sequence ELYK beginning at about amino acid 355 in a Net1 protein, where the amino acid immediately following the ELYK sequence is leucine. The location of the E385 residue in a Net1 protein can be determined by locating the amino acid sequence HYCK beginning at about amino acid 381 in a Net1 protein, where the amino acid immediately following the HYCK sequence is glutamic acid. The location of the T415 residue in a Net1 protein can be determined by locating the amino acid sequence AAGK beginning at about amino acid 411 in a Net1 protein, where the amino acid immediately following the AAGK sequence is threonine. The location of the 1452 residue in a Net1 protein can be determined by locating the amino acid sequence IAPC beginning at about amino acid 448 in a Net1 protein, where the amino acid immediately following the IAPC sequence is isoleucine.

A Net1 protein or a Net1 fragment may include an alteration of amino acid corresponding to L359, E385, T415, I452, or a combination thereof. The alteration may be a deletion of the amino acid, or a substation with a different amino acid. The substitution may be conservative or non-conservative. A Net1 protein that includes an alteration of one or more of those 4 amino acids may be a mature Net1 protein or any portion thereof, a Net1 fragment described herein, such as a Net1 fragment that includes CNLHARRCRFNMELYKLSGRKSGGVCLN (SEQ ID NO:16) or CNCNLHARRCRFNMELYKL SGRK SGGVCLNCRHNTAGRHCHYCKEGXYRDMGKPITH RKACKAC (SEQ ID NO:17), where the amino acid X is F or Y, optionally includes an alteration of the amino acid that corresponds to L359, E385 (amino acids 19 and 45, respectively, of SEQ ID NO17), or a combination thereof. Likewise, Net1 fragments having amino acids SEQ ID NO:17 and additional amino acids located on the carboxy-terminal end of the Net1 fragment may optionally include an alteration of the amino acid that corresponds to T415, I452, or a combination thereof In one embodiment, a Net1 protein having decreased ability to form a multimer, e.g., a_dimer, includes all or part of a V-2 region, all or part of a V-3 region, and may optionally include all or part of a V-1 region and all or part of a VI region. The ability of a Net1 protein having one or more of the alterations at amino acids corresponding to L359, E385, T415, I452, or a combination thereof, to form a multimer, e.g., a dimer, is relative to the ability of the same Net1 protein without the amino acid alterations.

The UNC5 binding domain and/or the DCC/NEO1 binding domain of a V-2 fragment or a V-2 region (or any other NET1 fragment described herein, including a fragment having a decreased ability to form a multimer), and/or a NET1 binding domain of a DCC/NEO1 fragment, and/or a Net1 protein having a decreased ability to form a multimer may include the conservative substitutions as described above, provided the binding domains can bind the appropriate ligand under suitable conditions. In one embodiment a Net1 fragment that includes CNLHARRCRFNMELYKLSGRKSGGVCLN (SEQ ID NO:16) or CNCNLHARRCRFNMELYKLSGRKSGGVCLNCRHNTAGRHCHYCKEGXYRDMGKPITH RKACKAC (SEQ ID NO:17) binds both an UNC5 protein and a DCC/NEO1 protein. The UNC5 binding domain and/or the DCC/NEO1 binding domain may include the non-conservative substitutions or deletions as described herein, thereby preventing binding of a binding domain to the appropriate ligand. Thus, in one embodiment a V-2 fragment or a V-2 region may bind an UNC5 protein and not bind a DCC/NEO1 protein, or may bind a DCC/NEO1 protein and not bind an UNC5 protein.

In one embodiment, in a NET1 fragment such as CNCNLHARRCRFNMELYKLSGRKSGGVCLNCRHNTAGRHCHYCKEGXYRDMGKPITH RKACKAC (SEQ ID NO:17) the cysteines at residues 1 and 10 are covalently bound by a di-sulfide bridge, the cysteines at residues 3 and 28 are covalently bound by a di-sulfide bridge, or a combination thereof.

A NET1 fragment also includes proteins that are structurally similar to the NET1 fragments described herein, and a DCC/NEO1 fragment also includes proteins that are structurally similar to the DCC/NEO1 fragments described herein. As used herein, a protein is “structurally similar” to a reference protein if the amino acid sequence of the protein possesses a specified amount of similarity and/or identity compared to the reference protein. Structural similarity of two proteins can be determined by aligning the residues of the two proteins (for example, a candidate protein and a reference protein such as amino acids CNLHARRCRFNMELYKLSGRKSGGVCLN (SEQ ID NO:16), or CNCNLHARRCRFNMELYKLSGRKSGGVCLNCRHNTAGRHCHYCKEGXYRDMGKPITH RKACKAC (SEQ ID NO:17), 946-956 of SEQ ID NO:3 (MVTKNRRSSTW), and the other amino acid sequences described herein) to optimize the number of identical amino acids along the lengths of their sequences; gaps in either or both sequences are permitted in making the alignment in order to optimize the number of identical amino acids, although the amino acids in each sequence must nonetheless remain in their proper order.

Unless modified as otherwise described herein, a pair-wise comparison analysis of amino acid sequences can be carried out using the Blastp program of the blastp suite-2sequences search algorithm, as described by Tatiana et al., (FEMS Microbiol Lett, 174, 247-250 (1999)), and available on the National Center for Biotechnology Information (NCBI) website. The default values for all blastp suite-2sequences search parameters may be used, including general paramters: expect threshold=10, word size=3, short queries=on; scoring parameters: matrix =BLOSUM62, gap costs=existence:11 extension:1, compositional adjustments=conditional compositional score matrix adjustment. Alternatively, proteins may be compared using the BESTFIT algorithm in the GCG package (version 10.2, Madison Wis.).

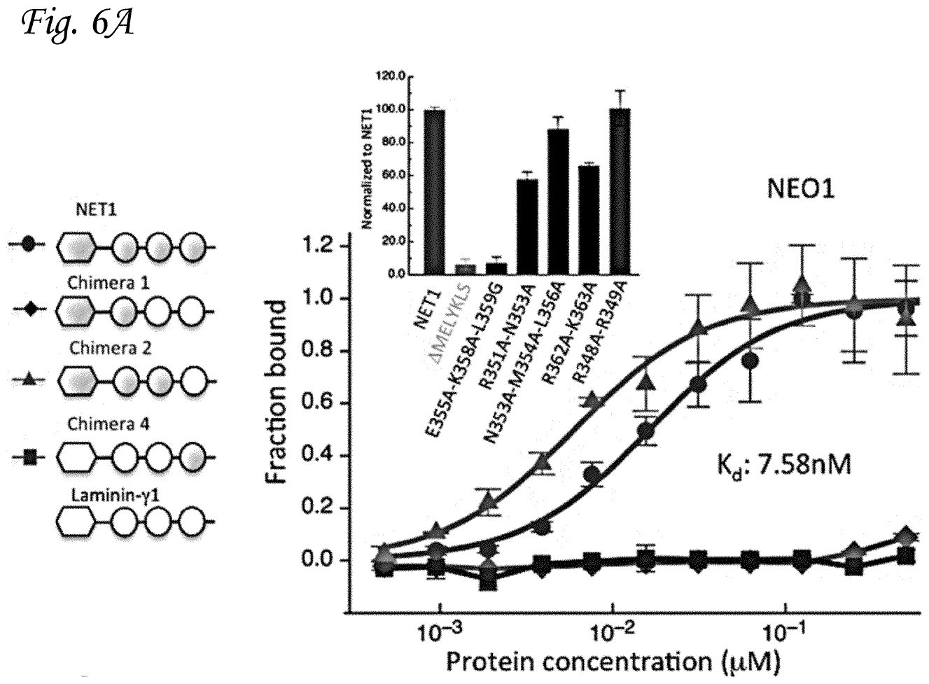

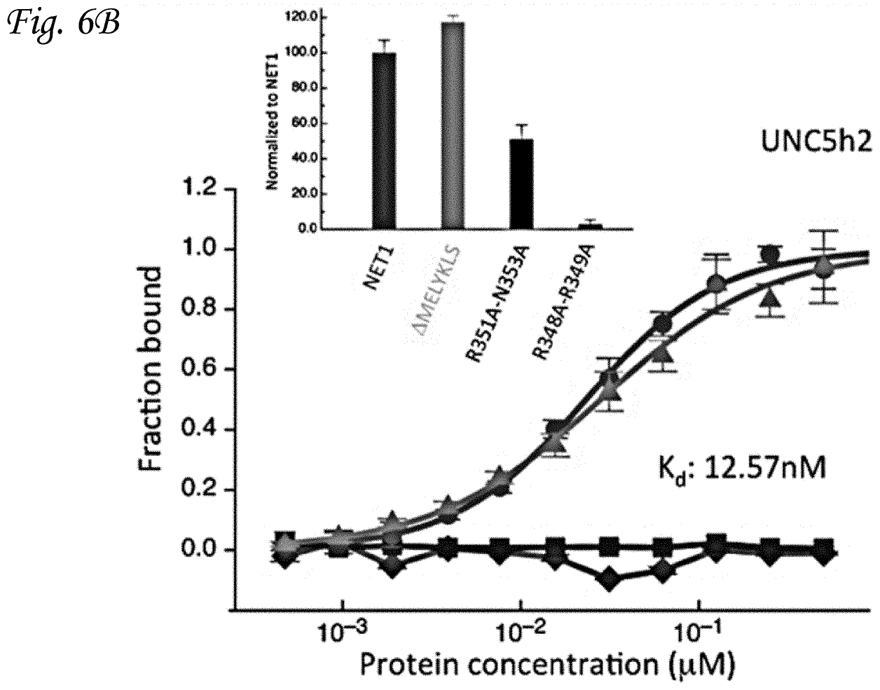

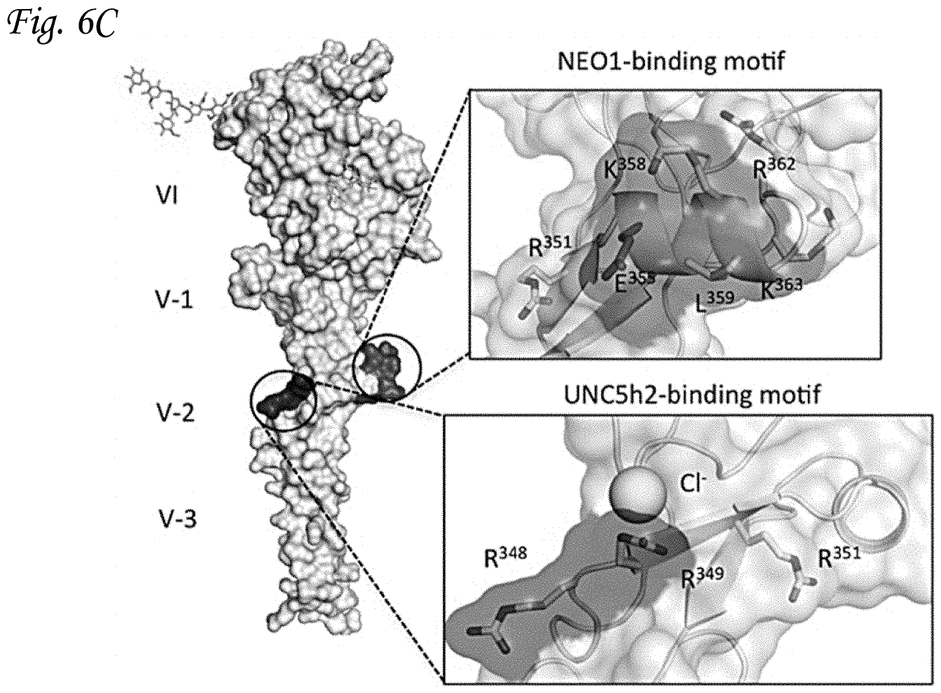

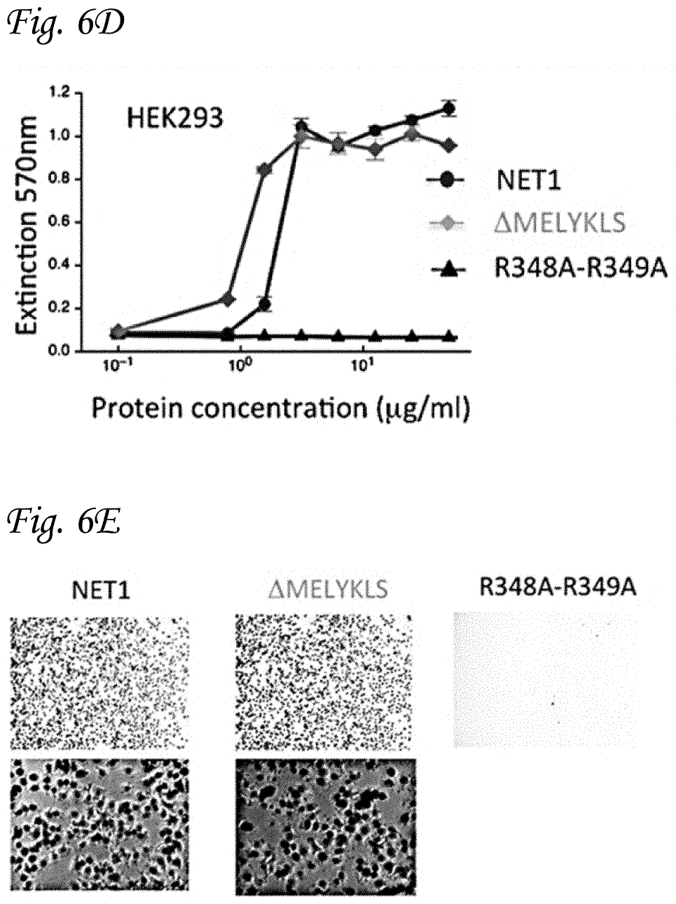

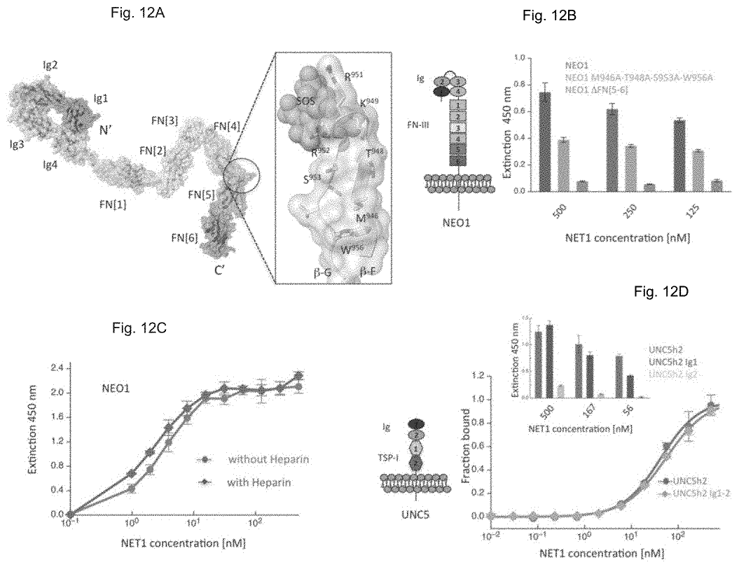

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. The skilled person will recognize that the structures of the mature NET1, the V-2 region, and the V-2 fragment (see FIGS. 1 and 6 of Example 1) can be used to help predict which amino acids may be substituted, and which sorts of substitutions (e.g., conservative or non-conservative) can be made to a NET1 fragment without altering the ability of the protein to bind UNC5 and/or DCC/NEO1. Likewise, the skilled person will recognize that the structures of the mature NEO1 (see FIG. 12) can be used to help predict with a reasonable expectation which amino acids may be substituted, and which sorts of substitutions (e.g., conservative or non-conservative) can be made to a DCC/NEO1 fragment without altering the ability of the protein to bind NET1. For instance, the skilled person can determine which amino acids are present on the surface of the protein, and will be able to predict with a reasonable expectation that certain non-conservative substitutions to those amino acids will not decrease binding of the protein. Likewise, the skilled person can determine which amino acids are present inside the protein. With this information the skilled person will be able to predict with a reasonable expectation that certain non-conservative substitutions to those amino acids will decrease binding of the protein. The skilled person will also be able to predict with a reasonable expectation that certain conservative substitutions to those amino acids will not decrease binding of the protein.

In one embodiment, a protein having an activity described herein can include a protein with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to a reference amino acid sequence.

In one embodiment, a protein having an activity described herein can include a protein with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a reference amino acid sequence.

As described herein a NET1 fragment, a DCC/NEO1 fragment, and a Net1 protein having a decreased ability to form a multimer may include a heterologous amino acid sequence. A heterologous amino acid sequence may be useful for purification of the NET1 fusion protein or a NEO1 fusion protein by affinity chromatography. Various methods are available for the addition of such affinity purification moieties to proteins. Representative examples include His-tags and maltose binding protein tags, and may be found in Hopp et al. (U.S. Pat. No. 4,703,004), Hopp et al. (U.S. Pat. No. 4,782,137), Sgarlato (U.S. Pat. No. 5,935,824), and Sharma (U.S. Pat. No. 5,594,115). In another example, the additional amino acid sequence may be a carrier protein. The carrier protein may be used to increase the immunogenicity of the fusion protein to increase production of antibodies that specifically bind to a NET1 fragment or a DCC/NEO1 fragment described herein. Examples of carrier proteins include, but are not limited to, keyhole limpet hemacyanin, bovine serum albumin, ovalbumin, mouse serum albumin, rabbit serum albumin, and the like. In another example, a NET1 fragment or a DCC/NE1 fragment may include amino acids that fluoresce, such as green fluorescent protein and derivatives thereof, and cytostatic agents, such as cholera toxin. In another example, a NET1 fragment or a DCC/NEO1 fragment may include a Fc region from an antibody.

Proteins described herein can be produced using recombinant DNA techniques, such as an expression vector present in a cell. Such methods are routine and known in the art. The proteins may also be synthesized in vitro, e.g., by solid phase peptide synthetic methods. The solid phase peptide synthetic methods are routine and known in the art. A protein produced using recombinant techniques or by solid phase peptide synthetic methods can be further purified by routine methods, such as fractionation on immunoaffinity or ion-exchange columns, ethanol precipitation, reverse phase HPLC, chromatography on silica or on an anion-exchange resin such as DEAE, chromatofocusing, SDS-PAGE, ammonium sulfate precipitation, gel filtration using, for example, Sephadex G-75, or ligand affinity. Such methods may also be used to isolate a NET1 fragment from a cell.

Whether a NET1 fragment described herein can bind to an UNC5 protein and/or a DCC/NEO1 protein can be determined using routine methods known to the skilled person. Whether a NET1 protein or a NET1 fragment described herein has a decreased ability to form a multimer, e.g., a dimer, with another Net1 protein and/or a Net1 fragment can be determined using routine methods known to the skilled person. An example of a UNC5 protein includes the UNC5 protein depicted at Genbank accession number CAD32251 (SEQ ID NO:7), such as the extracellular domain UNC5h2 IgG1-2 (amino acids 27-247 of the sequence depicted at Genbank accession no. CAD32251), or UNC5h2 IgG1 (amino acids 52-153 of the sequence depicted at Genbank accession no. CAD32251). An example of a NEO1 protein includes the NEO1 protein depicted at Genbank accession number AAH54540 (SEQ ID NO:3), such as the ectodomain of NEO1 depicted at amino acids 856-1054 of the sequence depicted at Genbank accession number AAH54540 (SEQ ID NO:3).

Whether a DCC/NEO1 fragment described herein can bind to a NET1 protein can also be determined using routine methods known to the skilled person. An example of a NET1 protein includes the mature NET1 protein depicted at Genbank accession number AAC52971 (SEQ ID NO:1). Other examples include the NET1 fragments described herein. Methods for detecting binding of a NET1 fragment to either UNC5 protein or DCC/NEO1 protein, and for detecting binding of a DCC/NEO1 fragment to NET1, include, for instance, solution binding assays and solid phase binding assays as shown in the examples.

### Antibody

Also provided herein are antibodies that specifically bind to a NET1 fragment. In one embodiment, an antibody specifically binds to an epitope present in the amino acid sequence HARRCR (SEQ ID NO:9). Such an antibody inhibits binding of an UNC5 protein to an UNC5 binding domain of a mature NET1 protein and a NET1 fragment. In another embodiment, an antibody specifically binds to an epitope present in the amino acid sequence MELYKLS (SEQ ID NO:10). Such an antibody inhibits binding of a DCC protein or a NEO1 protein to a DCC/NEO1 binding domain of a mature NET1 protein and a NET1 fragment. In one embodiment, an antibody specifically binds to an epitope present in amino acids of SEQ ID NO:16 (a V-2 fragment). Such an antibody may inhibit binding of an UNC5 protein to an UNC5 binding domain of a mature NET1 protein and a NET1 fragment, inhibit binding of a DCC protein or a NEO1 protein to a DCC/NEO1 binding domain of a mature NET1 protein and a NET1 fragment, or a combination thereof. In one embodiment, an antibody specifically binds to an epitope present in amino acids SEQ ID NO:17 (the V-2 region). Such an antibody may inhibit binding of an UNC5 protein to an UNC5 binding domain of a mature NET1 protein and a NET1 fragment, inhibit binding of a DCC protein or a NEO1 protein to a DCC/NEO1 binding domain of a mature NET1 protein and a NET1 fragment, or a combination thereof.

Further provided are antibodies that specifically bind a DCC/NEO1 fragment. In one embodiment, an antibody specifically binds to an epitope present in the amino acid sequence MVTK(N/G)RRSSTWS (SEQ ID NO:12). Such an antibody inhibits binding of a mature NET1 protein and, in some embodiments, a NET1 fragment to a NET1 binding domain of a NEO1 protein.

The antibody may be polyclonal or monoclonal, or a fragment thereof, e.g., scFv, Fab, F(ab′)2 or Fv. Laboratory methods for producing, characterizing, and optionally isolating polyclonal and monoclonal antibodies are known in the art and are routine (see, for instance, Harlow E. et al. Antibodies: A laboratory manual Cold Spring Harbor Laboratory Press, Cold Spring Harbor (1988). For instance, a NET1 fragment described herein can be administered to an animal in an amount effective to cause the production of antibody specific for the administered protein. Examples of animals that can be used for antibody production include mammals (e.g., mouse, rat, rabbit, Llama) and avail species. Optionally, a Net1 fragment is mixed with an adjuvant, for instance Freund's incomplete adjuvant, to stimulate the production of antibodies upon administration, or the NET1 fragment is fused to an immunogenic carries. Methods for identifying a monoclonal antibody that binds a specific epitope (such as an epitope present in HARRCR (SEQ ID NO:9) or MELYKLS (SEQ ID NO:10) or MVTK(N/G)RRSSTWS (SEQ ID NO:12)) are known to the skilled person and are routine.

### Compositions

Also provided herein are compositions that include a NET1 fragment, a DCC/NEO1 fragment, a NET1 protein having a decreased ability to form a multimer with another Net1 protein and/or a Net1 fragment, or an antibody described herein. A composition may include a pharmaceutically acceptable carrier. As used herein “pharmaceutically acceptable carrier” includes saline, solvents, dispersion media, coatings, antibacterial and antifungal agents, isotonic and absorption delaying agents, and the like, compatible with pharmaceutical administration. Additional active compounds can also be incorporated into the compositions.

A composition may be prepared by methods known in the art of pharmacy. In general, a composition can be formulated to be compatible with its intended route of administration. A formulation may be solid or liquid. Administration may be systemic or local. In some aspects local administration may have advantages for site-specific, targeted disease management. Local therapies may provide high, clinically effective concentrations directly to the treatment site, with less likelihood of causing systemic side effects.

Examples of routes of administration include parenteral (e.g., intravenous, intradermal, subcutaneous, intraperitoneal, intramuscular), enteral (e.g., oral or rectal), and topical (e.g., epicutaneous, inhalational, transmucosal) administration. Appropriate dosage forms for enteral administration of the compound of the present invention may include tablets, capsules or liquids. Appropriate dosage forms for parenteral administration may include intravenous administration. Appropriate dosage forms for topical administration may include nasal sprays, metered dose inhalers, dry-powder inhalers or by nebulization.

Solutions or suspensions can include the following components: a sterile diluent such as water for administration, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; electrolytes, such as sodium ion, chloride ion, potassium ion, calcium ion, and magnesium ion, and agents for the adjustment of tonicity such as sodium chloride or dextrose. pH can be adjusted with acids or bases, such as hydrochloric acid or sodium hydroxide. A composition can be enclosed in ampoules, disposable syringes or multiple dose vials made of glass or plastic.

Compositions can include sterile aqueous solutions or dispersions and sterile powders for the extemporaneous preparation of sterile solutions or dispersions. For intravenous administration, suitable carriers include physiological saline, bacteriostatic water, Cremophor EL™ (BASF, Parsippany, N.J.) or phosphate buffered saline. A composition is typically sterile and, when suitable for injectable use, should be fluid to the extent that easy syringability exists. It should be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile solutions can be prepared by incorporating the active compound (e.g., a NET1 fragment, a DCC/NEO1 fragment, a NET1 protein having a decreased ability to form a multimer with another Net1 protein and/or a Net1 fragment, or an antibody described herein) in the required amount in an appropriate solvent with one or a combination of ingredients such as those enumerated above, as required, followed by filtered sterilization. Generally, dispersions are prepared by incorporating the active compound into a sterile vehicle, which contains a dispersion medium and other ingredients such as from those enumerated above. In the case of sterile powders for the preparation of sterile injectable solutions, methods of preparation that may be used include vacuum drying and freeze-drying which yields a powder of the active ingredient plus any additional desired ingredient from a previously sterile-filtered solution thereof.

Oral compositions may include an inert diluent or an edible carrier. For the purpose of oral therapeutic administration, the active compound can be incorporated with excipients and used in the form of tablets, troches, or capsules. Oral compositions can also be prepared using a fluid carrier. Pharmaceutically compatible binding agents can be included as part of the composition. The tablets, pills, capsules, troches and the like may contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the active compounds may be delivered in the form of an aerosol spray, a nebulizer, or an inhaler, such as a nasal spray, metered dose inhaler, or dry-powder inhaler.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds may be formulated into ointments, salves, gels, or creams as generally known in the art. An example of transdermal administration includes iontophoretic delivery to the dermis or to other relevant tissues.

The active compounds can also be prepared in the form of suppositories (e.g., with conventional suppository bases such as cocoa butter and other glycerides) or retention enemas for rectal delivery.

The active compounds may be prepared with carriers that will protect the compound against rapid elimination from the body, such as a controlled release formulation, including implants. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially. Liposomal suspensions can also be used as pharmaceutically acceptable carriers. These can be prepared according to methods known to those skilled in the art. Delivery reagents such as lipids, cationic lipids, phospholipids, liposomes, and microencapsulation may also be used.

Toxicity and therapeutic efficacy of such active compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED50. Compounds which exhibit high therapeutic indices are preferred.

The data obtained from cell culture assays and animal studies can be used in formulating a range of dosage for use in humans. The dosage of such active compounds lies preferably within a range of concentrations that include the ED₅₀ with little or no toxicity. The dosage may vary within this range depending upon the dosage form employed and the route of administration utilized. For an active compound used in the methods of the invention, it may be possible to estimate the therapeutically effective dose initially from cell culture assays. A dose may be formulated in animal models to achieve a concentration range that includes the IC₅₀ (i.e., the concentration of the test compound which achieves a half-maximal inhibition of signs and/or symptoms) as determined in cell culture. Such information can be used to more accurately determine useful doses in humans.

The compositions can be administered one or more times per day to one or more times per week, including once every other day. The skilled artisan will appreciate that certain factors may influence the dosage and timing required to effectively treat a subject, including but not limited to the severity of a disease or disorder, previous treatments, the general health and/or age of the subject, and other diseases present. Moreover, treatment of a subject with an effective amount of an active agent can include a single treatment or can include a series of treatments.

### Methods of Use

Provided herein are methods for using the proteins described herein, including NET1 proteins, NET1 fragments, the DCC/NEO1 fragments, and the antibodies described herein. In one embodiment, a NET1 fragment may be used to inhibit binding of UNC5, DCC1, NEO1, or a combination thereof, to a NET1 protein. In one embodiment, a DCC/NEO1 fragment may be used to inhibit binding of NET1 protein to a NEO1 protein and/or a DCC protein. In one embodiment, an antibody that specifically binds an UNC5 binding domain HARRCR (SEQ ID NO:9) of a NET1 protein may be used to prevent binding of NET1 to UNC5. In one embodiment, an antibody that specifically binds a DCC/NEO1 binding domain MELYKLS (SEQ ID NO:10) of a NET1 protein may be used to prevent binding of NET1 to DCC, NEO1, or the combination thereof. In one embodiment, an antibody that specifically binds a NET1 binding domain MVTK(N/G)RRSSTWS (SEQ ID NO:12) of a NEO1 protein may be used to prevent binding of NEO1 to NET1.

The methods include contacting a protein with a fragment or an antibody described herein under conditions suitable for interaction between the the fragment or antibody with the protein. The protein, for instance, UNC5, DCC, NEO1, or NET1, may be in solution or cell associated. For instance, NET1 is typically present in an animal in solution and may be in solution when used in the methods. UNC5, DCC, and NEO1 are typically associated with the surface of a cell in an animal and may be associated with a surface when used in the methods. The surface may be a cell or an artificial surface. In other embodiments, an UNC5, DCC, and/or NEO1 may be in solution.

In one embodiment, a NET1 protein or NET1 fragment that includes an alteration of amino acid L359, E385, T415, and/or 1452 may be used to inhibit the formation of a dimer by a mature NET1 protein. This method includes contacting a NET1 protein with a NET1 protein or NET1 fragment that includes an alteration of amino acid L359, E385, T415, and/or 1452.

The contacting may be in vitro, ex vivo, or in vivo. As used herein, “in vitro” refers to a cell-free environment and to processes or reactions that occur within such an environment. An in vitro environment can be, but is not limited to, a test tube. As used herein, “ex vivo” refers to a cell that has been removed from the body of an animal. Examples of an animal include, but are not limited to, a human and a mouse. Ex vivo cells include, for instance, primary cells (e.g., cells that have recently been removed from a subject and are capable of limited growth in tissue culture medium), and cultured cells (e.g., cells that are capable of long term culture in tissue culture medium). The term “in vivo” refers to within the body of a subject. Examples of a subject include, but are not limited to, a human and a mouse

Another method of using the fragments and antibodies described herein is for inducing apoptosis of a cell. In certain cells dependence receptors play a role in regulating apoptosis of a cell (Mehlen and Bredesen, 2011, Sci. Signal., 4, mr2). For instance, DCC is a receptor that can induce apoptosis when NET1 is absent (Castets et al., 2012, Nature, 482:534). Likewise, in certain cells NEO1 and/or UNC5 are receptors that can induce apoptosis when NET1 is absent. Without intending to be limited by theory, it is believed that a dimeric NET1 binds to two receptors, e.g., DCC, NEO1, and/or UNC5 in any combination, or a single NET1 binds UNC5 and either NEO1 or DCC (see FIG. 16F), and causes the cross-linking of two dependence receptors. The cross-linking of the dependence receptors inhibits apoptosis and promotes cell survival. Inhibiting the cross-linking is predicted to trigger cell death through apoptosis.

In one embodiment, the method includes contacting a cell with a protein or an antibody that prevents the binding of NET1 to DCC, NEO1, UNC5, or a combination thereof. Such proteins include a NET1 fragment that binds DCC/NEO1 and/or UNC5. A NET1 fragment used in this method is preferably not a dimer. Another protein is a DCC/NEO1 fragment that binds NET1. Examples of antibody include antibody that specifically binds a DCC/NEO1 binding domain of a NET1 protein, antibody that specifically binds an UNC5 binding domain of a NET1 protein, and antibody that specifically binds a NET1 binding domain of a DCC/NEO1 protein.

In one embodiment, the method includes contacting a cell with a NET1 protein or NET1 fragment that inhibits the formation of multimers by NET1 proteins, e.g., a NET1 protein or NET1 fragment that includes an alteration of amino acid L359, E385, T415, and/or 1452.

The cells useful in this method are those that express DCC, NEO1, and/or UNC5 on the cell surface. Such a cell may be, but is not limited to, a cancer cell, such as colonic carcinoma, breast, prostate, adenocarcinoma, neuroblastoma, lung, as well as other cancer cells. The cell may be ex vivo or in vivo. In one embodiment, the method may include treating a subject having or at risk of having a cancer, wherein cells of the cancer express DCC, NEO1, and/or UNC5 on the cell surface. Examples of a subject include, but are not limited to, a human and a mouse.

Also provided herein are methods for identifying an agent that alters binding of a NET1 fragment or a DCC/NEO1 fragment to its ligand. In one embodiment, the method includes mixing a NET1 fragment, a ligand (e.g., an UNC5 protein, a DCC protein, or a NEO1 protein), and an agent together to form a mixture, and measuring the binding of the NET1 fragment to the ligand. A decrease of binding compared to a control mixture without the agent indicates the agent inhibits binding, and an increase of binding compared to a control mixture without the agent indicates the agent increases binding. The agent can be a chemical compound, including, for instance, an organic compound, an inorganic compound, a metal, a protein, a non-ribosomal protein, a polyketide, or a peptidomimetic compound. The sources for potential agents to be screened include, for instance, small molecule libraries, cell extracts of plants and other vegetations. Small molecule libraries are available, and include AMRI library, AnalytiCon, BioFocus DPI Library, Chem-X-Infinity, ChemBridge Library, ChemDiv Library, Enamine Library, The Greenpharma Natural Compound Library, Life Chemicals Library, LOPAC1280™ MicroSource Spectrum Collection, Pharmakon, The Prestwick Chemical Library®, SPECS, NIH Clinical Collection, and the Chiral Centers Diversity Library.

The present invention is illustrated by the following examples. It is to be understood that the particular examples, materials, amounts, and procedures are to be interpreted broadly in accordance with the scope and spirit of the invention as set forth herein.

### EXAMPLE 1

Netrin-1 (NET1) is a major guidance cue that plays a key role in diverse cellular processes including cell migration, adhesion, differentiation and survival. Here, we present the structure of NET1 and provide a molecular understanding for its unique complex formation with both neogenin NEO1 and UNC5. Two distinct binding epitopes at the V-2 subdomain are essential for mediating this interaction. The unique helical element in loop b associates with the fifth fibronectin domain of NEO1, whereas an arginine cluster in loop a binds to the first Ig domain of UNC5h2. The non-overlapping recognition sites allow for a simultaneous binding of NEO1 and UNC5. Whereas the cellular recognition of NET1 is primarily mediated via UNC5, neuronal branching is initialized by concurrent binding of NET1 to NEO1 and UNC5. Our findings reveal the molecular foundation of the bifunctional activity of NET1, which takes us a step forward in understanding its signaling activity.

Structural information on NET1 and its complexes with the dependence receptors NEO1/DCC and UNC5 is compulsory to obtain molecular insight into the mechanisms of signal transduction across the membrane. Here we present the crystal structure of glycoslylated NET1 at 2.6 A resolution. In addition, we provide a solution structure of the NEO1-NET1 complex determined by Small-angle X-ray scattering (SAXS). Together with functional data we offer mechanistic insights about how NET1 orchestrates NEO1/DCC and UNC5 binding.

A variety of different constructs were screened for expression and it was found that mouse NET1 composed of domains VI and V was expressed at high levels in its glycosylated form. This truncated form is advantageous for structural studies as it retains the ability to bind to the NET1- specific dependence receptors without forming the high molecular weight aggregates that are characteristic of the full-length protein (Wang et al., J Neurosci 19, 4938 (1999)). We determined the crystal structure of NET1 (FIG. 1) by multiple isomorphous dispersion (MAD) using Ta6Br12 as a heavy atom derivative (Table 1). Combined with anomalous dispersion from sulfur atoms (Table 2) we were able to confirm the amino acid register of NET1 and to localize individual disulfide bridges and the structural calcium (FIG. 2).

TABLE 1
Data collection, phasing, and refinement.

| | Native 1 | λ1 (peak) | λ2 (inflection) | λ3 (remote) |
|---|---|---|---|---|
| Data Collection | | | | |
| Wavelength (Å) | 1.2544 | 1.2547 | 1.2552 | 1.2410 |
| Resolution a*, b* (Å) | 44.85-2.8 | 44.74-3.47 | 44.76-3.48 | 48.95-3.52 |
| | (2.90-2.80) | (3.58-3.47) | (3.59-3.48) | (3.64-3.52) |
| Resolution c* (Å) | 44.85-2.64 | same as a* | same as a* | same as a* |
| | (2.74-2.64) | | | |
| Rₘₑₐₛ* | 0.133 (1.344) | 0.209 (0.541) | 0.168 (0.536) | 0.196 (0.687) |
| I/σI | 8.9 (2.0) | 4.2 (2.0) | 4.8 (2.0) | 4.5 (2.0) |
| Ellipsoidal | 1.000 (1.000) | — | — | — |
| Completeness | | | | |
| Spherical | 0.896 | 0.994 (1.000) | 0.995 (0.999) | 0.995 (1.000) |
| Completeness | | | | |
| Mean Redundancy | 10.6 (10.8) | 4.2 (3.9) | 4.2 (3.9) | 4.1 (4.0) |
| Cell dimensions | | | | |
| a = b, c (Å) | 69.75, 334.80 | 69.70, 333.26 | 69.72, 333.37 | 69.84, 333.39 |
| α, β, γ (°) | 90, 90, 120 | 90, 90, 120 | 90, 90, 120 | 90, 90, 120 |
| Phasing | | | | |
| Figure-of-merit* | | | | |
| (acentric/centric) | 0.296/0.292 | | | |
| Initial map vs final | 0.41 | | | |
| model* | | | | |
| Refinement | | | | |
| Resolution | 44.85-2.64 | | | |
| Number of reflections | 25768 (1308) [symbol] | | | |
| R_{work} (%)/R [symbol] (%) | 24.6/29.4 | | | |
| Mean B-factors (Å [symbol] ) | | | | |
| Protein/Ca²⁺/CT/H₂O | 52.9/38.8/19.8/37.1 | | | |
| Glycan at A [symbol] | 72.68 | | | |
| Glycan at A [symbol] | 94.71 | | | |
| Glycan at A [symbol] | 96.75 | | | |
| R.M.S.D. | | | | |
| Bond length (Å) | 0.014 | | | |
| Bond angles (°) | 1.697 | | | |
| Ramachandran [symbol] | | | | |
| Statistics [symbol] | | | | |
| Favoured (%) | 81.4 | | | |
| Allowed (%) | 18.6 | | | |
| Disallowed (%) | 0.0 | | | |

Statistics for the anomalous datasets (λ1-λ3) applies to separate Friedel mates I⁺/I⁻.
Numbers in parantheses represent the values for the highest ellipsoidal (Native 1) or spherical (λ1-λ3) resolution shell.
R.M.S.D. root m|ean square deviations
${}_{ }^{a}{R}{}_{meas}^{ } = \frac{{\sum }_{h}^{ } {( \frac{N}{N - 1} )}^{\frac{1}{2}} {\sum }_{i = 1}^{N} \vert I \text{?} - \overline{I} \text{?} \vert}{{\sum }_{h}^{ } {\sum }_{i = 1}^{N} I \text{?}}$ where N is the number of redundant observations for reflection h, Ī₍ₕ₎ the mean intensity of reflection h, and I₍ᵢₕ₎ the intensity of a redundant observation i for reflection h.
^{b}Figure of merit from 48.95 to 3.47 Å.
^{c}Averaged cosines of phase differences between initial map and phases generated from the final model from 44.85 to 2.64 Å.
^{d}Number of reflections in free set (5.1%)
^{e}R_{work} = Σₕₖₗ||F_{abs}|-|F [symbol] ||/Σₕₖₗ|F_{abs}|.
^{f}calculated using SwissModel and Procheck (1, 13). [symbol]
indicates data missing or illegible when filed

TABLE 2
Data collection for S-SAD.

| | S-SAD 1 | S-SAD 2 | S-SAD 3 | S-SAD 4 | S-SAD 5 |
|---|---|---|---|---|---|
| Data Collection | | | | | |
| Wavelength (Å) | 1.69950 | 1.69950 | 1.90745 | 1.90745 | 1.90745 |
| Resolution (Å) | 59.27-4.0 | 59.40-3.90 | 66.96-3.50 | 60.03-3.20 | 60.07-3.20 |
| | (4.22-4.00) | (4.11-3.90) | (3.83-3.50) | (3.42-3.20) | (3.42-3.20) |
| Rₘₑₐₛ* | 0.192 (0.482) | 0.152 (0.352) | 0.279 (0.889) | 0.209 (0.874) | 0.198 (0.864) |
| I/σI | 15.2 (7.7) | 19.3 (10.4) | 7.6 (2.8) | 7.2 (2.2) | 8.3 (2.6) |
| Completeness | 1.00 (1.00) | 0.990 (0.995) | 1.00 (1.00) | 1.00 (1.00) | 1.00 (1.00) |
| Redundancy | 11.6 (11.5) | 11.6 (11.5) | 7.4 (7.5) | 4.9 (5.0) | 6.7 (6.9) |
| Cell dimensions | | | | | |
| a = b, c (Å) | 69.54, 334.85 | 69.7, 335.4 | 69.45, 334.81 | 69.32, 334.75 | 69.36, 334.71 |
| α, β, γ (°) | 90, 90, 120 | 90, 90, 120 | 90, 90, 120 | 90, 90, 120 | |
| Phasing | | | | | |
| Number of DSB^{b} | 17 | | | | |
| Number of | 6 | | | | |
| Methionines | | | | | |
| Number of Ca²⁺ | 1 | | | | |
| Figure-of-merit | 0.300/0.173 | | | | |
| for S-SAD 1-5 | | | | | |
| (acentric/centric) | | | | | |
| Figure-of-merit* | 0.48 | | | | |
| (Initial map vs final | | | | | |
| model) | | | | | |
| Hand Score | | | | | |
| Correlation on \|E²\|/ | 3.25 (P3₂21) | | | | |
| Contrast of density | 1.57 (P3₁21) | | | | |
| modified map | | | | | |

Statistics for the anomalous datasets (S-SAD1-5) applies to separate Friedel mates I⁺/I⁻.
Numbers in parantheses represent the values for the highest resolution shell.
${}_{ }^{a}{R}{}_{meas}^{ } = \frac{{\sum }_{h}^{ } {( \frac{N}{N - 1} )}^{\frac{1}{2}} {\sum }_{i = 1}^{N} \vert I \text{?} - \overline{I} \text{?} \vert}{{\sum }_{h}^{ } {\sum }_{i = 1}^{N} I \text{?}}$ where N is the number of redundant observations for reflection h, Ī₍ₕ₎ the mean intensity of reflection h, and I₍ᵢₕ₎ the intensity of a redundant observation i for reflection h.
^{b}DSB Disulfide bridges
^{c}Averaged cosines of phase differences between initial map and phases generated from the final model from 44.85 to 2.64 Å.

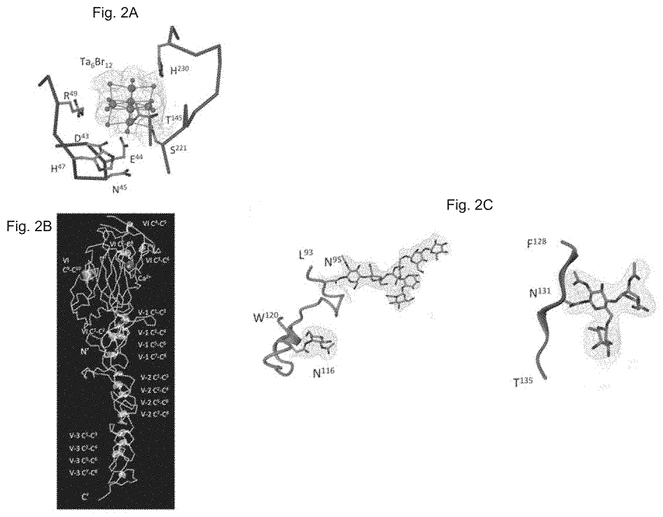

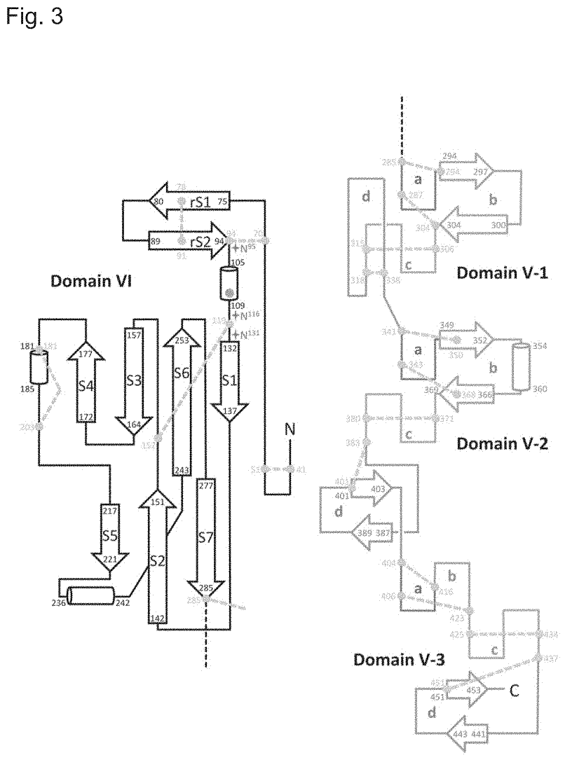

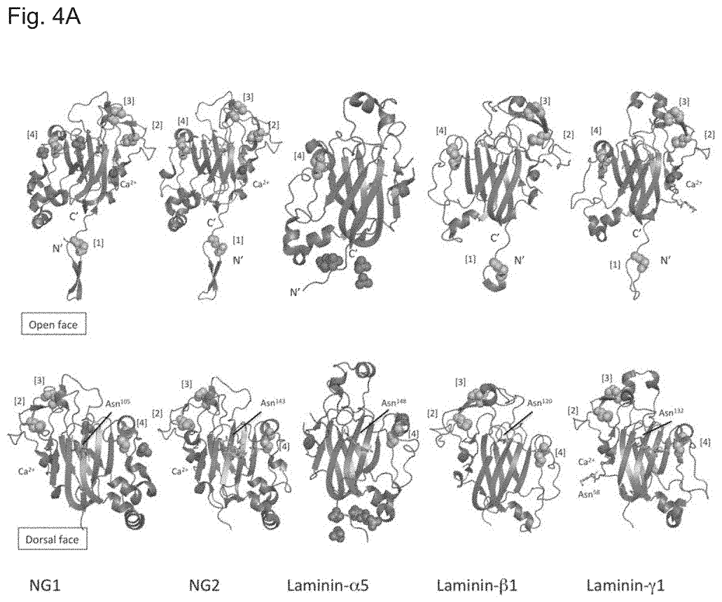

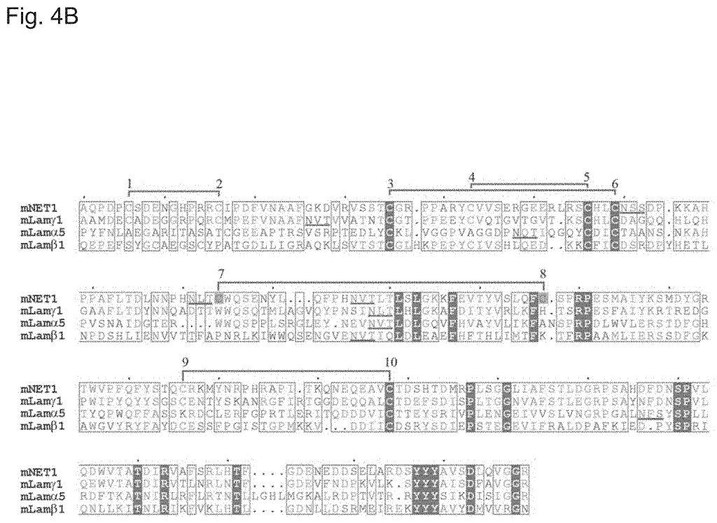

The structure of NET1 reveals a head to stalk arrangement with a length of 150 Å in which the globular shaped N-terminal domain VI forms the head and the three rod-like consecutive LE domains make up the stalk (FIG. 1A). The molecule has a total accessible surface area of about 23.000 Å² of which only 20% participate in inter-domain contacts. Domain VI forms a β-sandwich jelly-roll motif with a front face composed of sheet S5-S2-S7 and a dorsal face composed of sheet S4-S3-S6-S1 (FIG. 3). The topology of this domain is similar to the N terminal domain in laminin short arms and Netrin G proteins (Hussain et al., EMBO Rep 12, 276 (2011), Brasch et al., J Mol Biol 414, 723 (2011)) (FIG. 16). There is clear electron density for glycan additions at three asparagine-linked glycosylation sites, Asn95, Asn116, and Asn131 (FIGS. 1A and 2). These N-linked glycans create significant protrusions at the right hand of the apex of domain VI (Asn95 and Asn116) and at the dorsal face (Asn131). Whereas the latter is conserved in all secreted netrins and netrin Gs, Asn95 and Asn116 are unique to NET1 (Brasch et al., J Mol Biol 414, 723 (2011)). A calcium binding site is situated at the sugar-binding edge of the β- sandwich encompassing asparagine residues 95 and 116 (FIG. 1B). This structural calcium resides in a hidden cleft at the helical segment connecting the rim-sheet to β-strand S1 and is fixed by seven ligands forming a classical pentagonal bipyramid. Interestingly, the medial disulfide bridge between cysteine residues 119 and 152 that spans from S2 over S7 is in close spatial neighborhood to the structural calcium. Each of the three V subdomains adopt the classical LE-fold consisting of irregular coil segments and form a linear extended structure (Stetefeld et al., J Mol Biol 257, 644 (1996)). They are lacking a hydrophobic core, show few secondary structural elements and are stabilized by a characteristic pattern of conserved disulfide bridges. The most striking feature, however, is the highly electropositive nature of the V-2 subdomain (FIG. 1C). Cluster of arginine and lysine sidechains offer extended recognition sites for acidic ligands.

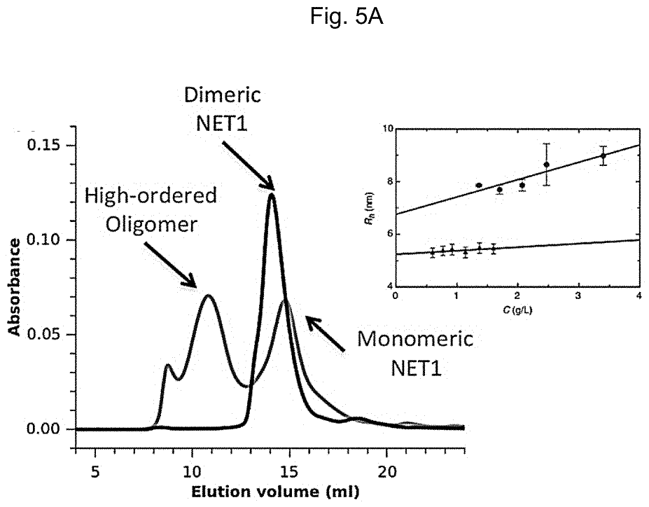

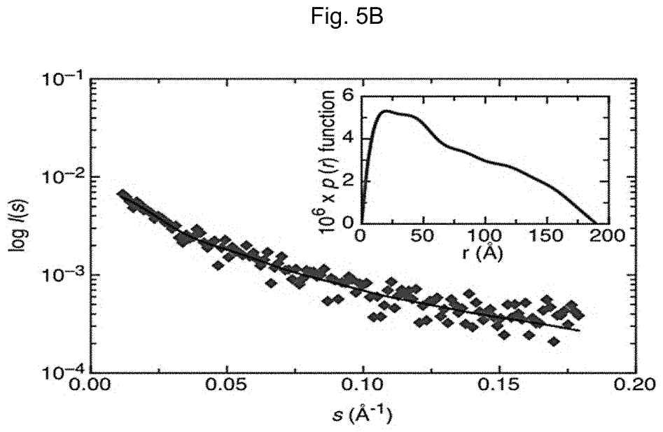

In solution, NET1 is able to self-associate and can assemble from monomeric to dimeric and to higher oligomeric forms (FIG. 5). In the presence of 1.0 M salt and 0.15 M glycine, NET1 adopts a stable dimeric entity (FIG. 1D). This differs markedly from its highly homologous counterpart, NET4, which is primarily monomeric in solution (Patel et al., Matrix Biol 31, 135 (2012)). A model of dimeric NET1 in solution was established from the ab initio SAXS envelope through superposition of the symmetric dimer within the NET1 crystal, followed by rigid body modelling (FIG. 5 and Table 3). Examination of the buried surface area (2.200 A²) revealed that only two contact points stabilize the dimeric NET1 assembly (FIG. 1D). Firstly, Leu359 from the helical segment of V-2 (site 1) forms van der Waals contacts with Ile452 from the antiparallel β-sheet of V-3. Secondly, in the center of gravity (site 2) Glu385 and Thr415 form a symmetric electrostatic cross over. Site 2 is reminiscent of a hinge between both monomers.

TABLE 3
SAXS data.

| | NET1 | | NEO1 | | NEO1 ΔFN[5-6] | | NEO-NET1 complex | |
|---|---|---|---|---|---|---|---|---|
| | Exp. | Hydropro- | Exp. | Hydropro- | Exp. | Hydropro- | Exp. | Hydropro- |
| | value | DAMMIN^{d} | value | BUNCH^{d} | value | CORAL^{d} | value | SASREF^{d} |
| Mw(Da)* | | | 117.6 | — | 92.50 | | 166.87 | |
| Rₕ (nm)^{a} | 5.24 ± 0.08 | 4.80 ± 0.05 | 7.06 ± 0.01 | 6.60 ± 0.04 | 6.42 ± 0.04 | 5.8 ± 0.1 | 8.65 ± 0.03 | 8.0 ± 0.06 |
| Rₕ (nm)^{b} | 4.7 ± 0.4 | | 7.0 ± 0.5 | | — | | 8.4 ± 0.5 | |
| R_{g} (nm)^{c} | 6.03 ± 0.02 | 6.00 ± 0.10 | 7.56 ± 0.02 | 7.44 ± 0.01 | 6.2 ± 0.05 | 5.93 ± 0.08 | 8.1 ± 0.08 | 8.3 ± 0.02 |
| Dₘₐₓ (nm)^{c} | 19.0 | 19.7 ± 0.02 | 23.8 | 24.0 ± 0.04 | 20.0 | 22.0 ± 0.13 | 24.8 | 25.8 ± 0.03 |
| χ value | 1.06 | | 0.9 | | 1.1 | | 1.0^{e} and 1.5^{f} | |
| NSD value | 0.36 ± 0.04 | | 2.8 ± 0.2 | | 2.1 ± 0.1 | | 0.5 ± 0.01 | |

*Calculated from amino acid sequence.
^{a}experimentally determined from DLS.
^{b}experimentally determined from SEC.
^{c}experimentally determined from P(y) analysis using GNOM.
^{d}SAXS model-based parameters calculated from HYDROPRO. Errors were calculated as standard deviation from multiple models.
^{e}values for NEO1 and
^{f}values for the NEO1-NET1 complex.

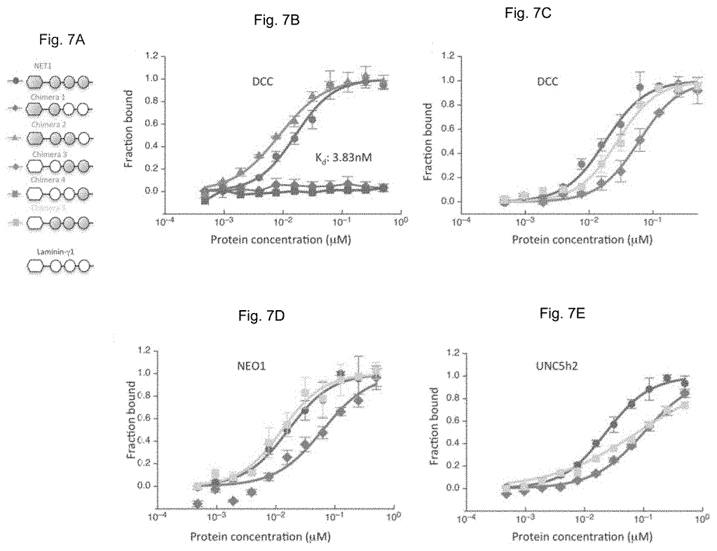

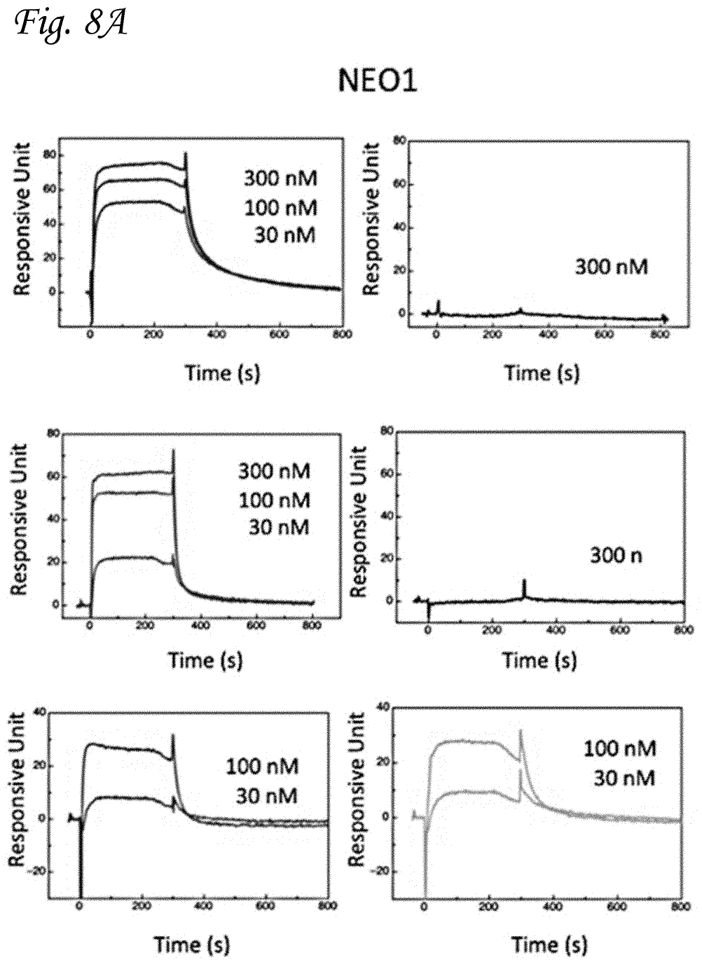

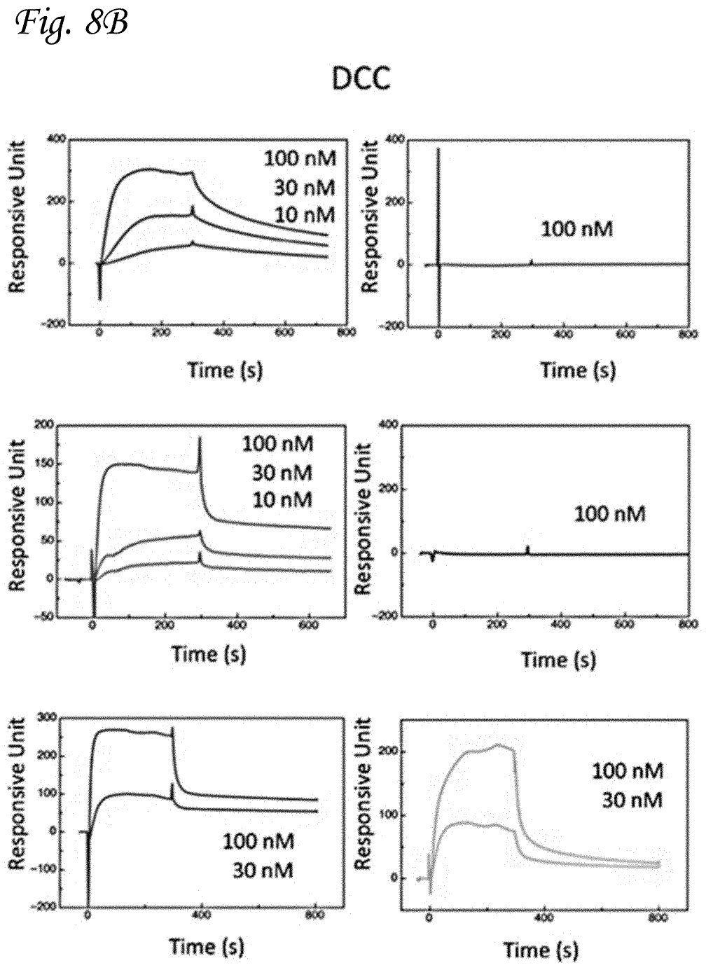

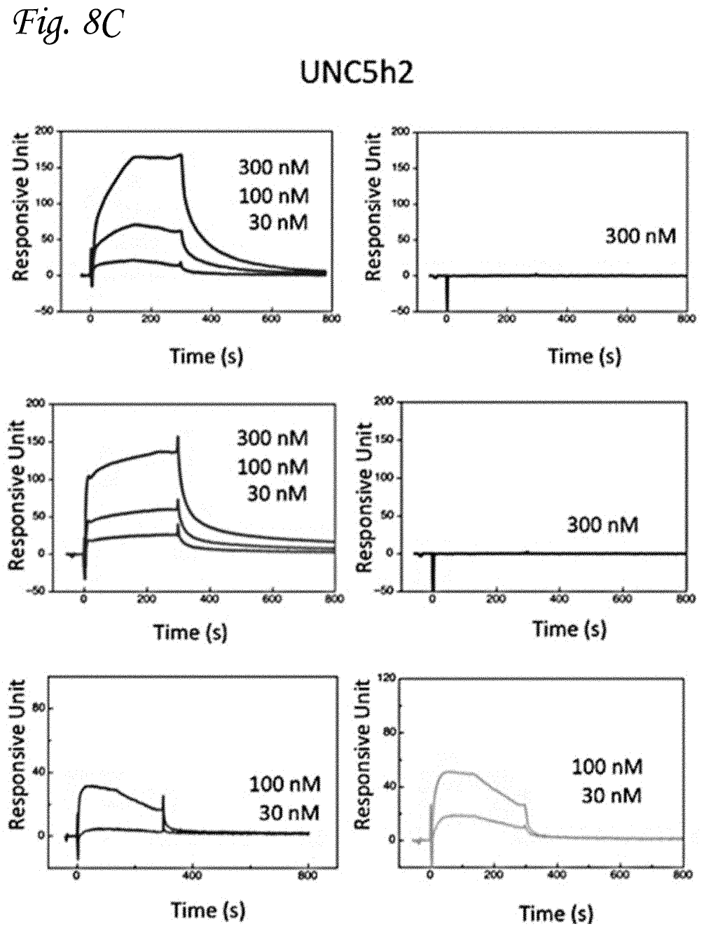

In an attempt to identify which domains of NET1 contained the dependence receptor binding epitopes, we generated a series of NET1 and laminin-γ1 short arm chimeras and evaluated their interaction with NEO1/DCC and UNC5h2 (FIGS. 2 and 7-8). Using this approach we took advantage of the fact that NET1 and laminin-γ1 both share the same modular topology, but they do not overlap in their binding specificity (Koch et al., J Cell Biol 151, 221 (2000), Yurchenco et al., Curr Opin Cell Biol 16, 572 (2004)). We have quantified the different binding profiles using a combination of solid-phase binding and surface plasmon resonance (SPR) techniques (FIGS. 7-8). Our results unambiguously assign subdomain V-2 of NET1 as the key site for binding to both NEO1/DCC and UNC5h2. The V-2 subdomain is the most highly conserved among the netrins and exhibits the highest similarity to the secreted NET3 (FIG. 9). Overall, the V domains of NET1 are most similar to the LE-repeats of the laminin γ1-subunit, with the exception of V-2 that is more closely related to the V-2 domain of the laminin β1- subunit (Lim et al., J Neurosci 22, 7080 (2002)). Beside NET1, only NET3 binds to the known dependence receptors of the DCC (NEO1 and DCC) and the UNC5 families (Wang et al., J Neurosci 19, 4938 (1999)).

To identify which individual amino acid residues are responsible for mediating the interaction with the full-length ectodomains of NEO1 and UNC5h2, we designed mutants taking into account the three-dimensional structure of V-2 and the degree of phylogenetic conservation (FIGS. 6 and 9). Clusters or patches of conserved amino acids on both, NET1 and NET3 surfaces are indicative for receptor binding sites. A structure guided deletion of the helical segment in loop b of V-2 (4354MELYKLS360 SEQ ID NO:10) led to an almost complete loss of binding to NEO1, whereas the UNC5h2 interaction was not affected (FIG. 6B). To analyze this primary site in more detail, we produced mutants that included changes in charge (E355A, K358A and R362A) and in hydrophobicity (L359G). Our results clearly indicate a major role for residues situated along the exposed face of the helix in loop b (FIG. 6C). Residues Glu355, Leu359 and Lys363 form the lower rim of the recognition site, whereas Lys358 and Arg362 flank it from above (FIG. 6C). To probe the UNC5h2 binding epitope, we swapped loop segments within the V-2 subdomain between NET1 and NET4 (FIG. 9). As a result of replacing the eight loop a/b residues 348RRCRFNME355 (SEQ ID NO:13) of NET1 with the equivalent residues from NET4 (339DTCHFDVN346 SEQ ID NO:14) we observed a 50-fold drop in affinity to UNC5h2, whereas NEO1 binding was unaffected. In a subsequent fine screen we observed an impairment of binding in the arginine double mutant (R348A-R349A) (FIG. 6B). Together Arg348 and Arg349 form a linear, solvent exposed patch of about 10 Å, which is flanked by Arg351 that precedes the helical element of loop b (FIG. 6C). Thus, we identified two adjacent but non-overlapping dependence receptor epitopes on the V-2 subdomain. Remarkably, the NEO1 -and the UNC5h2 binding epitopes are accessible in the dimeric NET1 entity in solution.

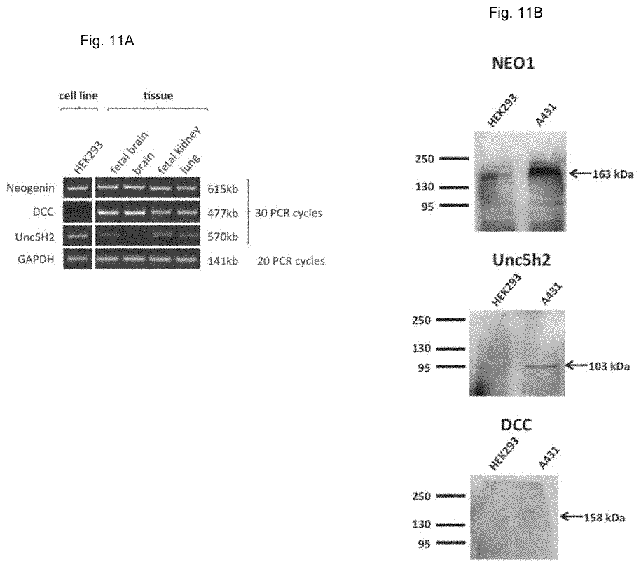

In a series of cell binding assays, we found that NET1 can act as adhesion molecule for NEO1 and UNC5 expressing cells (FIGS. 6D and E). Yebra et al. showed that full-length netrin-1 binds to pancreatic epithelial cells via an interaction mediated by domain C that is dependent on integrin (Yebra et al., Dev Cell 5, 695 (2003)). However, we discovered that NET1 lacking domain C can also act as a cell adhesion substrate for HEK293 cells. Interestingly, this phenomenon is independent of integrins because any addition of Mn2+ and Mg2+ or EDTA does not change the binding behavior (FIG. 10). The expression of NEO1 and UNC5h2 within HEK293 and melanoma A431 cells was confirmed by RT-PCR and western blot analysis (FIG. 11). In the next step, the respective binding partner on the cell surface was elucidated studying the Δ354MELYKLS360 (SEQ ID NO:10) truncation (NEO1 binding motif) and the R348A-R349A double mutant (UNC5h2 binding motif) of NET1. Our results identified UNC5h2 as a pivotal cell adhesion mediator of HEK293 cells to NET1 (FIGS. 2D and E). It is known that DCC transfected COS cells bind to NET1 in a long term adhesion (Ly et al., Cell 133, 1241 (2008)). Our discovery highlights UNC5 as the driving cell receptor, which mediates the initial step of NET1 binding. Additional secondary binding events to other receptors resulting in heteromerization are most likely to occur.

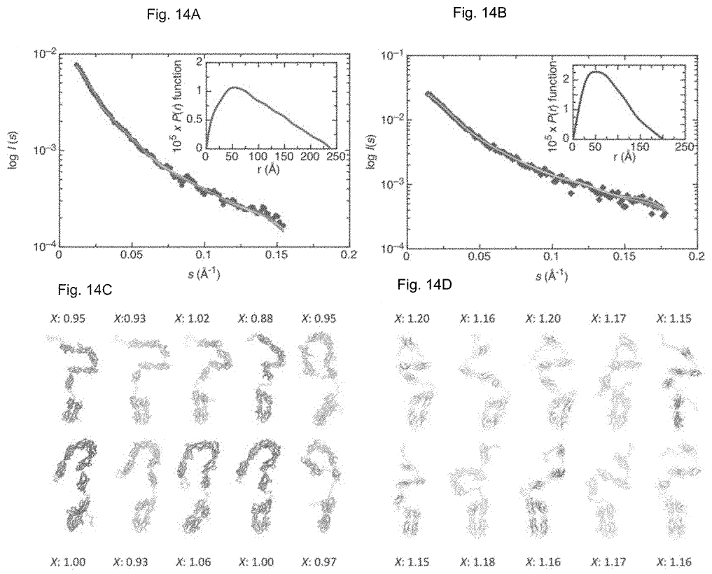

NEO1 and DCC are two vertebrate homologues that share approximately 50% amino acid identity and exhibit the same domain topology. In addition to their attractive roles in axon guidance, both receptors regulate cell-cell adhesion and tissue organization through their interactions with NET1 (Jarjour et al., J Neurosci 28, 11003 (2008)). Solution X-ray scattering revealed that the full-length ectodomain of NEO1 forms an extended S-shape with a Dmax of 240 Å (FIGS. 12A, 13 and 14). Fitting the high-resolution crystal structures for the Ig1-4 tandem (Chen et al., J Cell Sci 126, 186 (2013)), the individual FN domains [1-4] together with the FN[5-6] tandem (Yang et al., J Struct Biol 174, 239 (2010), Bell et al., Science 341, 77 (2013)) into the SAXS data, we were able to reconstruct the full-length ectodomain assembly of NEO1. The SAXS model consists of an N-terminal horseshoe including the four Ig domains that is followed by a serpentine arrangement of FN domains [1-6]. Structures of tandem FN domains have revealed a variety of quaternary arrangements, ranging from nearly linear (β4 integrin) to an overall spiral shape (NCAM, neuroglian and fibronectin) (Yang et al., J Struct Biol 174, 239 (2010)). Electron microscopy studies of full-length fibronectin clearly indicate several bend points with the observed conformation dependent upon ionic strength and the presence of acidic ligands (Engel et al., J Mol Biol 150, 97 (1981), Coles et al., Science 332, 484 (2011)). The overall flexibility of the FN[1-6] tandem of NEO1 might be a necessary feature for ligand binding and signaling complex formation.

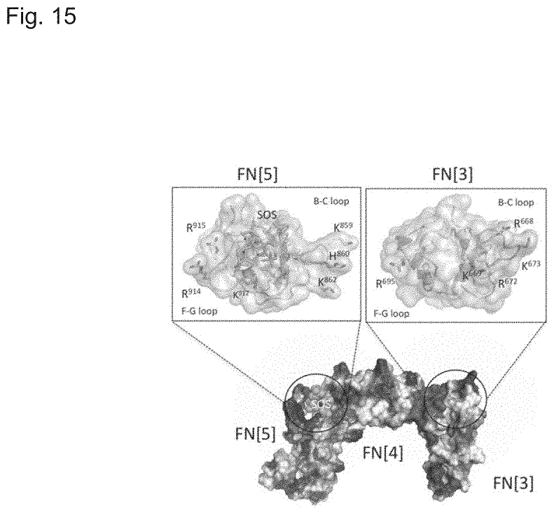

To decipher the precise NEO1 binding epitope for NET1, we compared the binding affinity of the full-length ectodomain of NEO1 with that of a truncated version (ΔFN[5-6]) (FIG. 12B). Our results demonstrate a key role for the FN[5] domain in NET1 binding, which is in agreement to previously published data (Geisbrecht et al., J Biol Chem 278, 32561 (2003)). Furthermore, we could show that four surface accessible amino acids that belong to the antiparallel β-sheet F-G of FN[5] form the NET1 recognition motif. The conserved residues Met946, Thr948 and Ser953 form an exposed pocket, which is flanked proximally by the indol ring of Trp956. The apical F-G loop segment is recruited by a cloud of positively charged residues (949KGRR952 SEQ ID NO:15) that are involved in heparin binding (Yang et al., J Struct Biol 174, 239 (2010), Bell et al., Science 341, 77 (2013)). In our NEO1 model, the surface exposed NET1 recognition motif is located directly at the interface between the FN[4] and FN[5] domains (FIG. 12A). It has been shown that a sucrose-octasulphate molecule (SOS) can adhere to the cleft between the F-G loop and the neighboring B-C loop segment of FN[5] (FIG. 15) (Bell et al., Science 341, 77 (2013)). Interestingly, mutagenesis studies of the KGRR (SEQ ID NO:15) motif to alanine hampered heparin binding, but resulted only in a marginal decrease in NET1 affinity (Geisbrecht et al., J Biol Chem 278, 32561 (2003)). This suggests that the basic edge of the F-G loop is not important for NET1 recognition. Our data demonstrate that heparin has no impact on the complex formation between NEO1 and NET1 (FIG. 12C). However, it is tempting to speculate that heparin binding regulates the conformational plasticity of the extended FN serpentine by stabilizing the elbow interface between FN[4-5] and FN[3-4], respectively (FIG. 15). Interestingly, heparan and diverse sulfate proteoglycans are known to regulate cell surface signaling events on neuronal extensions (Coles et al., Science 332, 484 (2011)). Binding of acidic ligands exerts effects on the quaternary arrangement and oligomerization of the receptor protein tyrosine phosphatase. Extracellularly, UNC5h2 is composed of an N-terminal Ig1-2 tandem followed by two thrombospondin type I (TSP-I) domains. We took a similar strategy to identify the NET1 binding epitope on the UNC5h2 receptor. Truncation experiments confirmed that the Ig1-2 tandem alone is sufficient for high-affinity NET1 binding (FIG. 12D) (Geisbrecht et al., J Biol Chem 278, 32561 (2003)). We demonstrate here that the Igl domain is the primary interaction domain for NET1. It is known that the Ig2 domain interacts with DSCAM and our results indicate that NET1 could induce UNC5/DSCAM heterodimerisation suggesting a supporting role for NET1 signaling through UNC5/DSCAM (Purohit et al., J Biol Chem 287, 27126 (2012)).

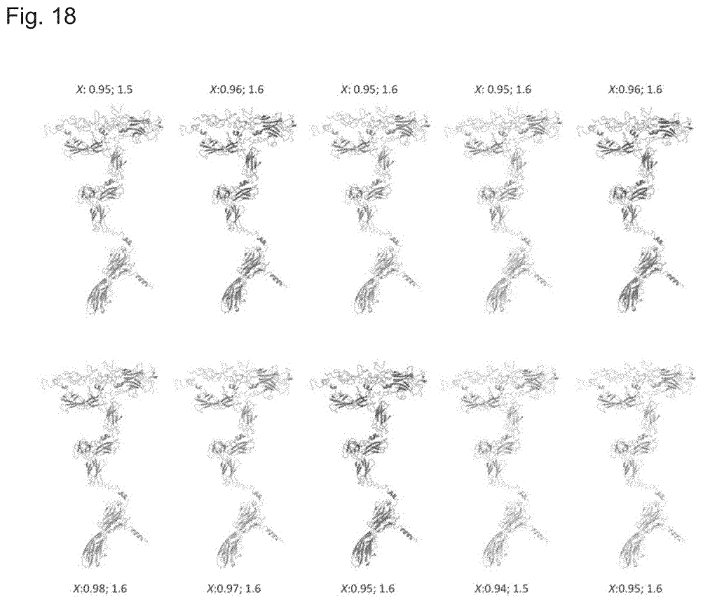

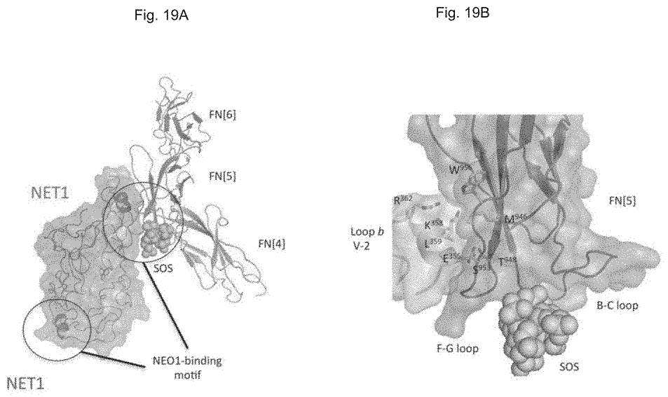

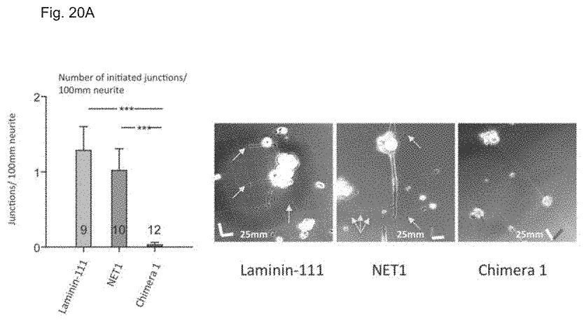

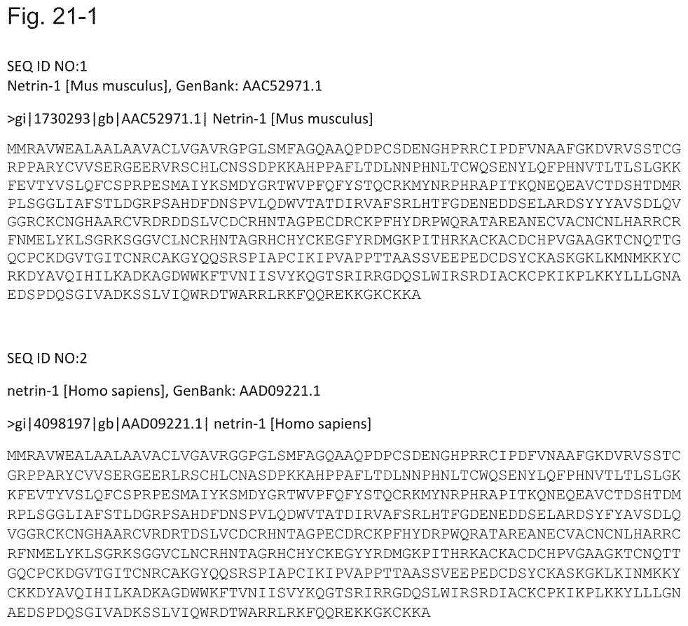

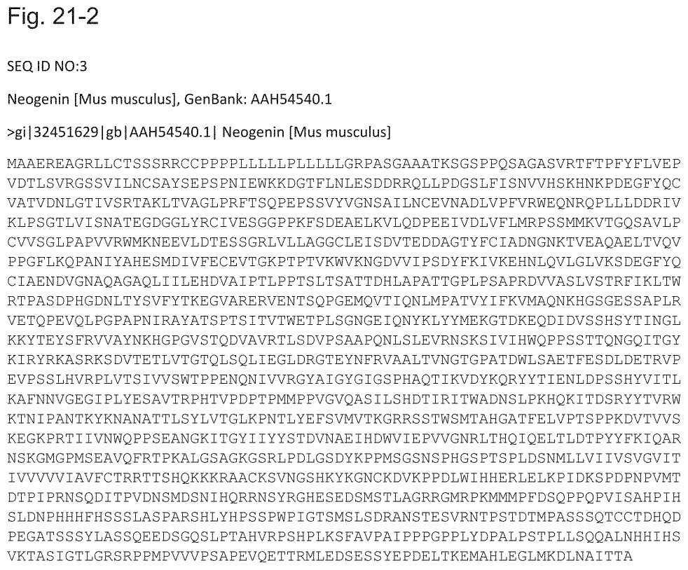

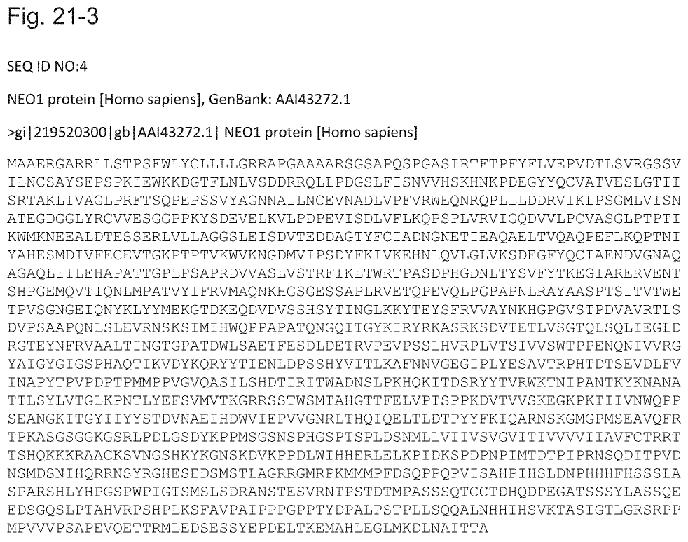

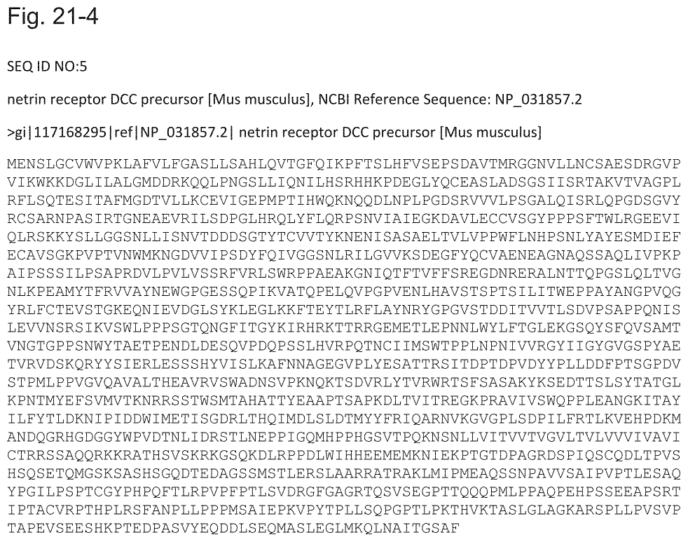

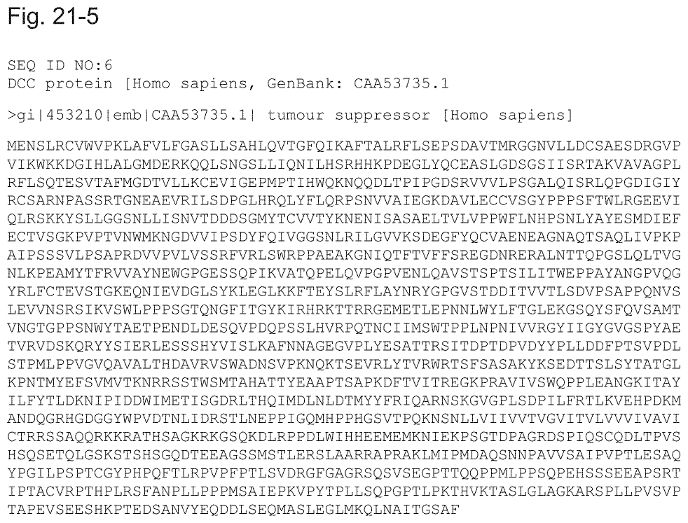

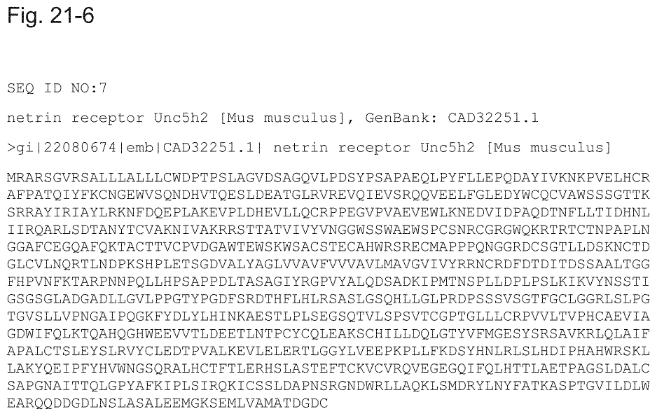

The complex between NEO1 and NET1 was purified by size exclusion chromatography and subsequently characterized by SAXS (FIGS. 16A&B and 17). The dependence receptor NEO1 forms a high-affinity interaction with NET1 in a 1:1 molar ratio. The V-2 domain of NET1 docks with its prominent, ridge-like protrusion formed by the unique a-helix in loop b to the concave surface of FN[5] that is shaped by the antiparallel β-sheet F-G (FIGS. 16C, 18, and 19). Despite the small buried surface area of about 900 Å², the interaction between NEO1 and NET1 is in the low nM range (FIG. 6). Interestingly, in the nidogen-laminin complex the LE domain ylIII-4 also forms a high-affinity complex with the nidogen domain G3, yet buries only 870 Å2 (FIG. 16C) (Takagi et al., Nature 424, 969 (2003)). Key residues in both LE repeats are oriented in a spatially similar manner (FIG. 16D). For example, residues Asp800, Asn802, and Val804 of laminin-γ1 bind to the concave surface at the top of the G3 β-propeller, which is similar to the Glu355, Lys358, Leu359 trio of NET1 that binds tightly to the top of FN[5]. It has been suggested that the rigidity of the nidogenlaminin interface minimizes the loss of entropy upon receptor-ligand binding and promotes the high-affinity interaction between both proteins (Takagi et al., Nature 424, 969 (2003)).

A key question in netrin-mediated signaling is how the axon guidance cue orchestrates the interaction with both, NEO1/DCC and UNC5 receptors. Our experiments reveal that two distinct, but spatially related, binding epitopes for NEO1/DCC and UNC5h2 exist on NET1. The flexible loops of the V-2 subdomain can be assigned to a mosaic of essentially discrete dependence receptor binding regions. To test the hypothesis of a concurrent receptor-binding mechanism, we studied the formation of a ternary complex between NET1, NEO1, and UNC5h2 (FIG. 16E). The binding of UNC5h2 to the NEO1-NET1 complex in the nanomolar range signifies simultaneous association of both receptors to NET1. Our results indicate a potential of NET1 to act as a molecular clamp with two different binding modes. Firstly, monomeric NET1 is able to form a ternary complex with NEO1/DCC and UNC5h2 in a 1:1:1 molar ratio (FIGS. 16F). Secondly, NEO1/DCC as well as UNC5 can bind dimeric NET1 to create a signaling complex of 2:2 stochiometry. Our data extend on the signaling hub mechanism (Bell et al., Science 341, 77 (2013)) by showing that NET1 mediated ligand-receptor clusters can be formed with a signaling compatible orientation relative to the cell surface. To explore the physiological relevance of our results we assessed the effect of V-2 modifications in neuronal branching experiments (FIG. 16G and 20). The assay indicates that binding of NEO1/DCC together with UNC5 is a prerequisite for NET1-mediated neuronal branching. It is well established that NET1 binding to NEO1/DCC mediates axon attraction whereas UNC5 is responsible for axon repulsion (Kennedy et al., Cell 78, 425 (1994), Serafini et al., Cell 87, 1001 (1996)). Our experiments implicate NEO1 and UNC5 as crucial receptors for branching initiation and it is only their simultaneous interaction with NET1 that leads to the activation signal.

### Materials and Methods

Recombinant expression and purification of NET1, UNC5h2, NEO1 and the NEO1-NET1 complex. NET1 from Mus musculus lacking the C-terminal domain (NP_032770, aa: 24-457) was cloned into a modified PCEP vector with an N-terminal octa-histidine (His8) tag. A pool of HEK293 cells were stably transfected followed by screening for high level expression. Secreted NET1 was purified by metal-affinity chromatography using nickel TALON beads (Clontech) followed by the removal of the His8 tag by thrombin digestion. The purified NET1 was then dialyzed against 50 mM Tris/Tris-HCl buffer pH 7.5 (supplemented with either 0.2 M NaCl or with 1.0 M NaCl plus 0.15 M glycine) and analyzed by size exclusion chromatography (SEC) on a Superdex 200 10/300 GL column using the AKTAFPLC system (GE Healthcare Life Sciences, USA). The elution was monitored by the absorption of ultraviolet light at 280 nm using a 0.5 mm flow cell. Similarly, the full-length ectodomain of NEO1 (AAH54540, aa: 41-1107) as well as a truncated form of the ectodomain of NEO1 (AAH54540, aa: 41-852) that lacked the terminal FN tandem [5-6] (ΔFN[5-6]) were cloned into PCEP vector system and fused to a C-terminal double Strep-tag II. The NEO1 ectodomains were expressed in stably transfected HEK293 cells and then purified by Streptavidin beads (IBA). A final purification step by SEC on the Superdex 200 10/300 GL column in 50 mM Tris/Tris-HCl, pH 7.5, 0.2 M NaCl was applied. The extracellular domains of UNC5h2 (CAD32251, aa: 25-374), UNC5h2 IgG1-2 (CAD32251, aa: 27-247), UNC5h2 IgG1 (CAD32251, aa: 52-153), and UNC5h2 IgG2 (CAD32251, aa: 154-245) carrying a C-terminal double Strep-tag II were cloned and expressed in stably transfected HEK293 cells followed by purification using Streptavidin beads. The NEO1-NET1 complex was assembled by incubating both species at room temperature for 30 minutes at equimolar concentrations in 50 mM Tris/Tris-HCl, pH 7.5, 0.2 M NaCl followed by purification of the complex via SEC on the Superdex 200 10/300 GL column. Fractions corresponding to the absorbance peaks in the elution profile were combined, concentrated and stored at 4° C. until required for experiments. Protein concentrations were based on calculated extinction coefficients of 46,475 M-1 cm-1 for NET1, 138,090 M-1 cm-1 for NEO1, 185,065 M-1 cm-1 for their complex and 104,210 M-1 cm-1 for the truncated version of NEO1 (ProtParam utility available on the ExPaSy server) (Gasteiger et al., Nucleic Acids Res 31, 3784 (2003)).

Design of chimeric constructs and site-directed mutagenesis. To identify the receptor binding sites in NET1, chimeric molecules were generated by overlap PCR using the Q5 (New England Biolabs) polymerase. The domains from NET1 (NET1: NP_032770) were replaced by the corresponding laminin-γ1sequences (laminin-γ1: NP_034813). The following constructs were amplified, cloned (with C-terminal Histidine tag), verified by sequencing, expressed, and purified: chimera 1 (NET1 aa: 25-340 fused to laminin-γ1 aa: 340-442), chimera 2 (NET1 aa: 25-403 fused to laminin-γ1 aa: 396-442), chimera 3 (laminin-γ1 aa: 34-395 fused to NET1 aa: 404-453), chimera 4 (laminin-γ1 aa: 34-339 fused to NET1 aa: 341-453), chimera 5 (laminin-γ1 aa: 34-283 fused to NET1 aa: 285-453), and laminin-γ1 as negative control (NP_034813, aa: 34-442). Furthermore, the following mutated NET1 and NEO1 molecules were generated: NET1: (NP_032770, aa: 1-457, R348A, R349A), (NP_032770, aa: 1-457, R351A, N353A), (NP_032770, aa: 1-457, R362A, K363A), (NP_032770, aa: 1-457, N353A, M354A, L356A), (NP_032770, aa: 1-457, 354MELYKLS360 SEQ ID NO:10), and (AAH54540, aa: 41-1107, M946A, T948A, S953A, W956).

Crystallization and data collection. NET1 was applied to a Superdex 200 SEC column (GE Healthcare) equilibrated with 50 mM Tris/Tris-HCl, pH 7.5, 0.2 M NaCl at room temperature and the collected peak fractions were immediately concentrated to 10 mg/ml and used for crystallization. NET1 crystals were grown by hanging-drop vapor diffusion at 293 K, by mixing equal volumes of protein and reservoir solution containing 0.1 M HEPES, pH 7.7, 2.7-2.8 M NaCl, 0.02-0.2 M glycine. Crystals appeared after 1 month and reached a final size of 0.1-0.5 mm after 3 months. NET1 crystals were soaked for 5-10 minutes in reconstituted crystallization mother solution containing 5% triethylene glycol before flash freezing in liquid nitrogen and data collection at 100 K. A high-resolution native dataset (Native 1) was collected at the CMCF-BM beamline at the Canadian Light Source, Saskatoon, Canada. A three-wavelengths anomalous derivative dataset (λ1-λ3) from a crystal soaked in Ta6Br12 for 48 hours prior to data collection was obtained on beamline X06SA at the Swiss Light Source, Villigen, Switzerland. Two native single wavelength anomalous datasets (S-SAD 1 and 2) for sulphur phasing were collected on beamline X06SA and another three (S-SAD3-5) were collected at beamline 23-ID-B of the Advanced Photon Source, Argonne National Laboratory, Argonne, USA. The native and all three TABR-MAD datasets were indexed, integrated and scaled with the HKL2000 suite (Otwinowski and Minor, Methods in Enzymology 276, 307 (1997)) and scalepack (Evans, Acta Crystallogr D Biol Crystallogr 62, 72 (2006)). The five sulfur-SAD datasets were processed with iMOSFLM (Battye et al., Acta Crystallogr D Biol Crystallogr 67, 271 (April 2011)) and the CCP4 package (Acta Crystallogr D Biol Crystallogr 50, 760 (1994)). The NET1 crystals grew in spacegroup P3221 with one molecule in the asymmetric unit. All crystals featured strong anisotropic diffraction, so we performed ellipsoidal truncation of the high resolution dataset using the program Anisotropyxfiles (Strong et al., Proc Natl Acad Sci USA 103, 8060 (2006)) and anisotropic scaling in Phaser (McCoy et al., J Appl Crystallogr 40, 658 (2007)). See Tables 1 and 2 for data statistics.

Structure determination and refinement. The structure was determined by multiple isomorphous replacement with anomalous scattering (MIRAS) using the peak (X1), inflection (λ2) and energy remote (λ3) datasets of a MAD experiment with Ta6Br12, and the native data set (Native 1). The single bound Ta6Br12 cluster could be located by Patterson methods in SHELXD (Sheldrick and Schneider, Methods Enzymol 277, 319 (1997)) and refined within SHARP (Bricogne et al., Acta Crystallogr D Biol Crystallogr 59, 2023 (2003)). The overall figure-of-merit for acentric and centric reflections were 0.296 and 0.292, respectively (Table 1). Phases were improved using the procedure provided by SHARP. Using the native and MAD datasets together with the correctly positioned Ta6Br12 coordinates, 20 cycles of solvent flipping in conjunction with initial density modification improved the figure of merit to 0.41. The highest |E2|/contrast was obtained by choosing a solvent content of 65%. The solvent mask was generated from a partial homology model generated in SwissModel (Arnold et al., Bioinformatics 22, 195 (2006)). The correlation on |E2|/contrast for the density modified map of 2.01 (P3121) vs 3.23 (P3221) allowed for an unambiguous assignment of the correct crystallographic spacegroup. The obtained experimental Fourier map was of excellent quality and secondary structure elements were recognized, showing continuous density for a large portion of the main chains, side chains and N-linked glycan linkages. An initial model of NET1 was built manually in COOT (Emsley et al., Acta Crystallogr D Biol Crystallogr 66, 486 (2010)), except in the region of the Ta6Br12 cluster, where the structure was perturbed. The register of all 17 disulfide bridges, the position of the six methionine residues and the structural calcium were determined using the anomalous signal of sulphur and calcium from the 1.90745 Å longwavelength datasets (Table 2). The best quality electron density map was generated in SHARP using the high-resolution dataset (Native 1) and all five long-wavelength datasets (S-SAD 1-5) simultaneously, employing the same phase improvement procedure as above, but using the current model to generate the solvent mask. This map was of unrivaled quality and allowed to complete the entire model. The structure was refined in REFMAC (Murshudov et al., Acta Crystallogr D Biol Crystallogr 67, 355 (2011)) and COOT. Refinement strategies included bulk solvent correction and anisotropic scaling of the data, individual coordinate refinement and TLS parameterization. Real-space refinement was performed in COOT into a maximum likelihood-weighted 2mFo-DFc map calculated in REFMAC. The final model was refined to 2.6 Å with Rwork and Rfree values of 24.6% and 29.4%. The quality of the structure was validated with SwissModel and PROCHECK (Gasteiger et al., Nucleic Acids Res 31, 3784 (2003), Laskowsky et al., J. Appl. Cryst 26, 283 (1993)). The final model comprises amino acid residues 36-456 except for residues 83-85 which are disordered. The model further contains one calcium ion, two chloride ions, one glycine and sixteen water molecules. The side chains of Asn95, Asn116 and Asn131 are glycosylated with the following partial glycan structures visible: α-D-mannose(1-3)[α-D-mannose(1-3)α-D-mannose(1-6)]β-D-mannose(1-4)β-D-N-acetyl glucosamine(1-4)β-D-N-acetyl glucosamine-Asn95; β-D-N-acetyl glucosamine-Asn116; β-D-Nacetyl glucosamine(1-4)[α-L-fucose(1-6)]β-D-N-acetyl glucosamine-Asn131.The carbohydrate structures were validated using PDB-CARE (Lutteke an d von der Lieth, BMC Bioinformatics 5, 69 (2004)) and CARP (Lutteke et al., Glycobiology 16, 71R (2006); Lutteke et al., Nucleic Acids Res 33, D242 (2005)). The refinement statistics can be found in Table 1. Interface contacts were determined using FastContact and electrostatic potentials were generated using APBS (Camacho and Zhang, Bioinformatics 21, 2534 (2005); Baker et al., Proc Natl Acad Sci USA 98, 10037 (2001)). Buried surface area values of protein-protein interactions were calculated using the PISA webserver for a probe radius of 1.4 A (Krissinel and Henrick, J Mol Biol 372, 774 (2007)). Structure figures were prepared using the program PyMOL and sequence conservation analysis was performed using MULTILAN (Corpet, Nucleic Acids Res 16, 10881 (1988)) and ESCRIPT (Gouet et al., Bioinformatics 15, 305 (1999)).

Biophysical characterization and Small Angle X-ray Scattering (SAXS) of NET1, NEO1 and the NEO1-NET1 complex. The size characteristics of the purified and concentrated NET1, NEO1, their complex and the truncated version of NEO1 were determined using a Nano-S Dynamic Light Scattering system (Malvern Instruments Ltd, Malvern, UK) used previously (Patel et al., Matrix Biol, (2013); Patel et al., Matrix Biol 29, 565 (2010); Patel et al., Matrix Biol, (2011)). All samples were filtered through a 0.1 μm filter (Millipore, USA) before dilution to yield a series of solutions at multiple concentrations. For each measurement, the protein was allowed to equilibrate for 4 minutes at 293 K prior to DLS measurements, after which multiple records of the DLS profile were collected for data analysis. SAXS data for dimeric NET1 were collected at 1.0 mg/ml in 50 mM Tris/Tris-HCl, pH 7.5 supplemented with 1.0 M NaCl and 0.15 M glycine using the S-Max3000 SAXS/WAXS (Rigaku instrument) as described previously (Patel et al., Matrix Biol 29, 565 (2010)). SAXS data for NEO1 (at 1.0, 2.0, 2.6, 3.2, 3.7, and 4.2 mg/ml), the truncated version of NEO1 (2.6, 3.1, 3.6, 4.6, and 5.1 mg/ml) and the NEO1-NET1 complex (0.8, 1.2, 1.6, and 2.0 mg/ml) were collected in 50 mM Tris/Tris-HCl, pH 7.5, 0.2 M NaCl. Primary data analysis was carried out in PRIMUS (Konarev et al., J Appl Crystallogr 36, 1277 (2003)) followed by the determination of the maximum particle dimension (Dmax) and Rg for each protein by GNOM analysis (Svergun, J Appl Crystallogr 25, 495 (1992)). Ab initio modelling was performed in DAMMIN (Svergun, Biophys J 76, 2879 (1999)), whereas the rigid body modelling was performed using the programs BUNCH, SASREF and CORAL (Petoukhov and Svergun, Biophys J 89, 1237 (2005), Petoukhov et al., J Appl Crystallogr 45, 342 (2012)). Solid Phase Binding Assay. Purified proteins were coated at 10 μg/ml (500 ng/well) overnight at 4° C. onto 96-well plates (Nunc Maxisorb). After washing with TBS, plates were blocked for 2 hours at room temperature with TBS containing 3% bovine serum albumin. Ligands were diluted to concentrations from 0.475 nM to 500 nM and incubated for 2 hours at room temperature. After extensive washing with TBS, bound ligands were detected either with an anti-His antibody (Qiagen; dilution 1:1000) for His-tagged proteins, followed by horseradish peroxidase (HRP)-conjugated porcine anti-mouse immunoglobulins (DAKO Cytomation) or via a streptavidin horseradish peroxidase (HRP)-conjugate (IBA; dilution 1:5000) for Strep-tagged proteins. HRP was detected by Pierce TMB ELISA Substrate (Thermo Scientific™). Absorption was measured at 450 nm after stopping the reaction with 2 M sulfuric acid.

Surface Plasmon Resonance Binding Assays. Assays were performed using a Biacore 2000 (BIAcore AB). Proteins were coupled to the CMS chip in 10 nM sodium acetate, pH 5.0 or 4.5, respectively, at a flow rate of 5 μl/min. The chip surface was previously activated with a 7 minutes pulse of 0.005 nm N-hydroxysuccinide and 1-ethyl-3-(3-dimethylamino-propyl) carbodimide hydrochloride. After coupling the required amount of protein (1000 RU), unbound reactive groups were saturated with 1 M ethanolamine hydrochloride, pH 8.5. The experiments were carried out using serial dilutions of putative binding partner. The analyte was passed over the sensor chip with a constant flow rate of 30 μl/min for 300 s, and dissociation was measured over 500 s. Fitting of the data, overlay plots, and calculation of kD values were done with BIAevaluation software 3.2 according to the Langmuir model for 1:1 binding.

Static cell adhesion assays. 96-well plates were coated overnight at 4° C. with a serial dilution (0-50 μg/ml) of recombinantly expressed NET1 and the respective NET1 mutants. HEK293 cells were trypsinized and washed with 1×PBS. Afterwards cells were resuspended in serum-free DMEM/F-12 and 5×104 cells/well were seeded in triplicates. Cells were allowed to adhere to the various substrates for 30 minutes at 37° C. After washing of non-adherent cells with 1×PBS, adherent cells were fixed with 1% glutaraldehyde for 15 minutes at room temperature before staining for 25 minutes with 0.1% crystal violet. Adherent cells were quantified by eluting the dye with 0.2% Triton X-100. The absorbance was measured in a spectrophotometer (Tecan) at 570 nm. A blank value corresponding to BSA coated wells was automatically subtracted.

Neurite outgrowth assay. Neurite outgrowth from acutely prepared dorsal root ganglia (DRG) neurons was assayed using microcontact-printed substrates. Namely, on coverslips patterned with recombinant laminin-111 (Biolamina, Stockholm, Sweden) and the NET1 variants below as the test substrate. Negative silicon masters used to create stamps for microcontact printing were provided by Dr. Siegmund Schroeter (Institute of Photonic Technology, Jena, Germany) from which PDMS stamps were cast, as described (Philipsborn et al., Development 133, 2487 (2006)). Protein printing inks were prepared containing 20 μg/ml recombinant laminin-111 or one of the NET1 variants (NET1 wild type, chimera 1, R348AR349A double mutant and 4354MELYKLS360 truncation) in TBS containing 1 mM CaCl2. The printing ink also contained 2 μg/ml Alexa 555-conjugated goat anti-rabbit antibodies to provide a fluorescent marker for detecting printed regions. Coverslips for stamping were cleaned as previously described and activated with oxygen plasma for 1 minute immediately prior to stamping. Stamps covered in printing ink were incubated at 37° C. for 2 hours, then rinsed with ultrapure water and dried with nitrogen. Immediately after drying, the protein was printed from the stamp onto freshly activated glass coverslips. To create a cross-patterned substrate, the test protein was always printed on the coverslip first, the coverslip was then carefully removed from the stamp, rotated through 90 degree and then the recombinant laminin-111 was printed in bands at right angles to the test protein structures, creating a grid pattern. As a positive control, recombinant laminin-111 was printed in both directions. The printed substrates were then used within 1 hour of printing.

Neuronal cell culture. Dorsal root ganglia (DRG) from Mus musculus (aged 4-6 weeks) were dissected and collected in 1 ml PBS on ice, then treated for 30 minutes at 37° C. with 1 μg/ml collagenase IV in 1 ml PBS and finally for 5-20 minutes at 37° C. with 0.05% trypsin in 1 ml PBS. Cells were dissociated in 1 ml DMEM/F-12 by being passaged through a 20 G needle then collected, washed and finally resuspended in D-MEM/F-12 medium containing 10% horse serum. Cells (about 60-120 μl of cell suspension per coverslip) were seeded on printed substrates. After 6 hours, an additional DRG medium was added to the coverslips. Cells were cultured for 24-48 hours at 37° C. in a Steri-Cult 200 incubator. No nerve growth factor or other neurotrophins were added to the medium. After neurite outgrowth over the recombinant laminin-111 was observed, cells were fixed using 4% PFA in PBS for 15 minutes at room temperature. Phase contrast images of the cells were obtained using an Axiovert 200, inverted light microscope (Zeiss, Jena, Germany). Fluorescence images of the labeled protein substrates were also obtained to visualize the protein pattern. Neurite outgrowth was assessed in two ways: First, the ratio of neurite outgrowth between test substrate and recombinant laminin-111 was calculated. Secondly, branching events where neurite branches had initiated on the test substrate, regardless of whether the neurite extended over the substrate or not, were counted and the number of branch points per unit length calculated. All data represents measurements obtained from cultures from at least 4 mice and at least 5 printed coverslips. The data were compared using the Student's t-test after verifying that they followed a normal distribution.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A NET1 fragment comprising an amino acid sequence having at least 80% identity to amino acids CNLHARRCRFNMELYKLSGRKSGGVCLN (SEQ ID NO:16),
wherein the NET1 fragment comprises an UNC5 binding domain HARRCR, wherein the UNC5 binding domain comprises conservative substitutions in HARRCR at positions 1, 2, 3, 4, 5, 6, or a combination thereof,
wherein the NET1 fragment comprises a DCC/NEO1 binding domain MELYKLS, wherein the DCC/NEO1 binding domain comprises conservative substitutions in MELYKLS at positions 1, 2, 3, 4, 5, 6, 7, or a combination thereof, and
wherein the NET1 fragment comprises UNC5 binding activity and DCC/NEO1 binding activity.

2. A DCC/NEO1 fragment comprising an amino acid sequence having at least 80% identity to amino acids MMPPVGVQASILSHDTIRITWADNSLPKHQKITDSRYYTVRWKTNIPANTKYKNANATT LSYLVTGLKPNTLYEFSVMVTKGRRSSTWSMTAHGATFELVP (SEQ ID NO:18) or MLPPVGVQAVALTHEAVRVSWADNSVPKNQKTSDVRLYTVRWRTSFSASAKYKSEDT TSLSYTATGLKPNTMYEFSVMVTKNRRSSTWSMTAHATTYEAAP (SEQ ID NO:19),
wherein the DCC/NEO1 fragment comprises a NET1 binding domain MVTK(N/G)RRSSTWS, wherein the NET1 binding domain comprises conservative substitutions in MVTK(N/G)RRSSTWS at positions 1, 2, 3, 4, 5, 6, 7, 8, 9, 10, 11, 12, or a combination thereof,
wherein the DCC/NEO1 fragment comprises NET1 binding activity.

3-6. (canceled)

7. A method for inhibiting binding of a NET1 protein to an UNC5 protein comprising contacting an UNC5 protein with the NET1 fragment of claim 1.

8. A method for inhibiting binding of a NET1 protein to a DCC/NEO1 protein comprising contacting a DCC protein with the NET1 fragment of claim 1.

9. A method for inhibiting binding of a DCC or a NEO1 protein to a NET1 protein comprising contacting a NET1 protein with the DCC/NEO1 fragment of claim 2.

10-18. (canceled)

19. A method for inducing apoptosis of a cell comprising:
contacting a cell with i) the NET1 fragment of claim 1,
wherein the cell is a cell that expresses a DCC protein, a NEO1 protein, an UNC5 protein, or a combination thereof, on the surface of the cell.

20. A method for inducing apoptosis of a cell comprising:
contacting a cell with i) the DCC/NEO1 fragment of claim 2,
wherein the cell is a cell that expresses a DCC protein, a NEO1 protein, or a combination thereof, on the surface of the cell.

21-28. (canceled)

29. A NET1 fragment that comprises an alteration of an amino acid corresponding to L359, E385, T415, 1452, or a combination thereof, of a NET1 protein, wherein the NET1 fragment will not form a multimer.

30. A method comprising contacting a cell with the NET1 fragment of claim 29.

31-38. (canceled)


SEQUENCE LISTING (31 sequences)

SEQ ID NO: 1 - protein, 603 aa, Mus musculus
MMRAVWEALAALAAVACLVGAVRGPGLSMFAGQAAQPDPCSDENGHPRRCIPDFVNAAFGKDVRVSSTCGRPPARYCVVSERGEERVRSCHLCNSSDPKKAHPPAFLTDLNNPHNLTCWQSENYLQFPHNVTLTLSLGKKFEVTYVSLQFCSPRPESMAIYKSMDYGRTWVPFQFYSTQCRKMYNRPHRAPITKQNEQEAVCTDSHTDMRPLSGGLIAFSTLDGRPSAHDFDNSPVLQDWVTATDIRVAFSRLHTFGDENEDDSELARDSYYYAVSDLQVGGRCKCNGHAARCVRDRDDSLVCDCRHNTAGPECDRCKPFHYDRPWQRATAREANECVACNCNLHARRCRFNMELYKLSGRKSGGVCLNCRHNTAGRHCHYCKEGFYRDMGKPITHRKACKACDCHPVGAAGKTCNQTTGQCPCKDGVTGITCNRCAKGYQQSRSPIAPCIKIPVAPPTTAASSVEEPEDCDSYCKASKGKLKMNMKKYCRKDYAVQIHILKADKAGDWWKFTVNIISVYKQGTSRIRRGDQSLWIRSRDIACKCPKIKPLKKYLLLGNAEDSPDQSGIVADKSSLVIQWRDTWARRLRKFQQREKKGKCKKA

SEQ ID NO: 2 - protein, 604 aa, Homo sapiens
MMRAVWEALAALAAVACLVGAVRGGPGLSMFAGQAAQPDPCSDENGHPRRCIPDFVNAAFGKDVRVSSTCGRPPARYCVVSERGEERLRSCHLCNASDPKKAHPPAFLTDLNNPHNLTCWQSENYLQFPHNVTLTLSLGKKFEVTYVSLQFCSPRPESMAIYKSMDYGRTWVPFQFYSTQCRKMYNRPHRAPITKQNEQEAVCTDSHTDMRPLSGGLIAFSTLDGRPSAHDFDNSPVLQDWVTATDIRVAFSRLHTFGDENEDDSELARDSYFYAVSDLQVGGRCKCNGHAARCVRDRTDSLVCDCRHNTAGPECDRCKPFHYDRPWQRATAREANECVACNCNLHARRCRFNMELYKLSGRKSGGVCLNCRHNTAGRHCHYCKEGYYRDMGKPITHRKACKACDCHPVGAAGKTCNQTTGQCPCKDGVTGITCNRCAKGYQQSRSPIAPCIKIPVAPPTTAASSVEEPEDCDSYCKASKGKLKINMKKYCKKDYAVQIHILKADKAGDWWKFTVNIISVYKQGTSRIRRGDQSLWIRSRDIACKCPKIKPLKKYLLLGNAEDSPDQSGIVADKSSLVIQWRDTWARRLRKFQQREKKGKCKKA

SEQ ID NO: 3 - protein, 1465 aa, Mus musculus
MAAEREAGRLLCTSSSRRCCPPPPLLLLLPLLLLLGRPASGAAATKSGSPPQSAGASVRTFTPFYFLVEPVDTLSVRGSSVILNCSAYSEPSPNIEWKKDGTFLNLESDDRRQLLPDGSLFISNVVHSKHNKPDEGFYQCVATVDNLGTIVSRTAKLTVAGLPRFTSQPEPSSVYVGNSAILNCEVNADLVPFVRWEQNRQPLLLDDRIVKLPSGTLVISNATEGDGGLYRCIVESGGPPKFSDEAELKVLQDPEEIVDLVFLMRPSSMMKVTGQSAVLPCVVSGLPAPVVRWMKNEEVLDTESSGRLVLLAGGCLEISDVTEDDAGTYFCIADNGNKTVEAQAELTVQVPPGFLKQPANIYAHESMDIVFECEVTGKPTPTVKWVKNGDVVIPSDYFKIVKEHNLQVLGLVKSDEGFYQCIAENDVGNAQAGAQLIILEHDVAIPTLPPTSLTSATTDHLAPATTGPLPSAPRDVVASLVSTRFIKLTWRTPASDPHGDNLTYSVFYTKEGVARERVENTSQPGEMQVTIQNLMPATVYIFKVMAQNKHGSGESSAPLRVETQPEVQLPGPAPNIRAYATSPTSITVTWETPLSGNGEIQNYKLYYMEKGTDKEQDIDVSSHSYTINGLKKYTEYSFRVVAYNKHGPGVSTQDVAVRTLSDVPSAAPQNLSLEVRNSKSIVIHWQPPSSTTQNGQITGYKIRYRKASRKSDVTETLVTGTQLSQLIEGLDRGTEYNFRVAALTVNGTGPATDWLSAETFESDLDETRVPEVPSSLHVRPLVTSIVVSWTPPENQNIVVRGYAIGYGIGSPHAQTIKVDYKQRYYTIENLDPSSHYVITLKAFNNVGEGIPLYESAVTRPHTVPDPTPMMPPVGVQASILSHDTIRITWADNSLPKHQKITDSRYYTVRWKTNIPANTKYKNANATTLSYLVTGLKPNTLYEFSVMVTKGRRSSTWSMTAHGATFELVPTSPPKDVTVVSKEGKPRTIIVNWQPPSEANGKITGYIIYYSTDVNAEIHDWVIEPVVGNRLTHQIQELTLDTPYYFKIQARNSKGMGPMSEAVQFRTPKALGSAGKGSRLPDLGSDYKPPMSGSNSPHGSPTSPLDSNMLLVIIVSVGVITIVVVVVIAVFCTRRTTSHQKKKRAACKSVNGSHKYKGNCKDVKPPDLWIHHERLELKPIDKSPDPNPVMTDTPIPRNSQDITPVDNSMDSNIHQRRNSYRGHESEDSMSTLAGRRGMRPKMMMPFDSQPPQPVISAHPIHSLDNPHHHFHSSSLASPARSHLYHPSSPWPIGTSMSLSDRANSTESVRNTPSTDTMPASSSQTCCTDHQDPEGATSSSYLASSQEEDSGQSLPTAHVRPSHPLKSFAVPAIPPPGPPLYDPALPSTPLLSQQALNHHIHSVKTASIGTLGRSRPPMPVVVPSAPEVQETTRMLEDSESSYEPDELTKEMAHLEGLMKDLNAITTA

SEQ ID NO: 4 - protein, 1450 aa, Homo sapiens
MAAERGARRLLSTPSFWLYCLLLLGRRAPGAAAARSGSAPQSPGASIRTFTPFYFLVEPVDTLSVRGSSVILNCSAYSEPSPKIEWKKDGTFLNLVSDDRRQLLPDGSLFISNVVHSKHNKPDEGYYQCVATVESLGTIISRTAKLIVAGLPRFTSQPEPSSVYAGNNAILNCEVNADLVPFVRWEQNRQPLLLDDRVIKLPSGMLVISNATEGDGGLYRCVVESGGPPKYSDEVELKVLPDPEVISDLVFLKQPSPLVRVIGQDVVLPCVASGLPTPTIKWMKNEEALDTESSERLVLLAGGSLEISDVTEDDAGTYFCIADNGNETIEAQAELTVQAQPEFLKQPTNIYAHESMDIVFECEVTGKPTPTVKWVKNGDMVIPSDYFKIVKEHNLQVLGLVKSDEGFYQCIAENDVGNAQAGAQLIILEHAPATTGPLPSAPRDVVASLVSTRFIKLTWRTPASDPHGDNLTYSVFYTKEGIARERVENTSHPGEMQVTIQNLMPATVYIFRVMAQNKHGSGESSAPLRVETQPEVQLPGPAPNLRAYAASPTSITVTWETPVSGNGEIQNYKLYYMEKGTDKEQDVDVSSHSYTINGLKKYTEYSFRVVAYNKHGPGVSTPDVAVRTLSDVPSAAPQNLSLEVRNSKSIMIHWQPPAPATQNGQITGYKIRYRKASRKSDVTETLVSGTQLSQLIEGLDRGTEYNFRVAALTINGTGPATDWLSAETFESDLDETRVPEVPSSLHVRPLVTSIVVSWTPPENQNIVVRGYAIGYGIGSPHAQTIKVDYKQRYYTIENLDPSSHYVITLKAFNNVGEGIPLYESAVTRPHTDTSEVDLFVINAPYTPVPDPTPMMPPVGVQASILSHDTIRITWADNSLPKHQKITDSRYYTVRWKTNIPANTKYKNANATTLSYLVTGLKPNTLYEFSVMVTKGRRSSTWSMTAHGTTFELVPTSPPKDVTVVSKEGKPKTIIVNWQPPSEANGKITGYIIYYSTDVNAEIHDWVIEPVVGNRLTHQIQELTLDTPYYFKIQARNSKGMGPMSEAVQFRTPKASGSGGKGSRLPDLGSDYKPPMSGSNSPHGSPTSPLDSNMLLVIIVSVGVITIVVVVIIAVFCTRRTTSHQKKKRAACKSVNGSHKYKGNSKDVKPPDLWIHHERLELKPIDKSPDPNPIMTDTPIPRNSQDITPVDNSMDSNIHQRRNSYRGHESEDSMSTLAGRRGMRPKMMMPFDSQPPQPVISAHPIHSLDNPHHHFHSSSLASPARSHLYHPGSPWPIGTSMSLSDRANSTESVRNTPSTDTMPASSSQTCCTDHQDPEGATSSSYLASSQEEDSGQSLPTAHVRPSHPLKSFAVPAIPPPGPPTYDPALPSTPLLSQQALNHHIHSVKTASIGTLGRSRPPMPVVVPSAPEVQETTRMLEDSESSYEPDELTKEMAHLEGLMKDLNAITTA

SEQ ID NO: 5 - protein, 1447 aa, Mus musculus
MENSLGCVWVPKLAFVLFGASLLSAHLQVTGFQIKPFTSLHFVSEPSDAVTMRGGNVLLNCSAESDRGVPVIKWKKDGLILALGMDDRKQQLPNGSLLIQNILHSRHHKPDEGLYQCEASLADSGSIISRTAKVTVAGPLRFLSQTESITAFMGDTVLLKCEVIGEPMPTIHWQKNQQDLNPLPGDSRVVVLPSGALQISRLQPGDSGVYRCSARNPASIRTGNEAEVRILSDPGLHRQLYFLQRPSNVIAIEGKDAVLECCVSGYPPPSFTWLRGEEVIQLRSKKYSLLGGSNLLISNVTDDDSGTYTCVVTYKNENISASAELTVLVPPWFLNHPSNLYAYESMDIEFECAVSGKPVPTVNWMKNGDVVIPSDYFQIVGGSNLRILGVVKSDEGFYQCVAENEAGNAQSSAQLIVPKPAIPSSSILPSAPRDVLPVLVSSRFVRLSWRPPAEAKGNIQTFTVFFSREGDNRERALNTTQPGSLQLTVGNLKPEAMYTFRVVAYNEWGPGESSQPIKVATQPELQVPGPVENLHAVSTSPTSILITWEPPAYANGPVQGYRLFCTEVSTGKEQNIEVDGLSYKLEGLKKFTEYTLRFLAYNRYGPGVSTDDITVVTLSDVPSAPPQNISLEVVNSRSIKVSWLPPPSGTQNGFITGYKIRHRKTTRRGEMETLEPNNLWYLFTGLEKGSQYSFQVSAMTVNGTGPPSNWYTAETPENDLDESQVPDQPSSLHVRPQTNCIIMSWTPPLNPNIVVRGYIIGYGVGSPYAETVRVDSKQRYYSIERLESSSHYVISLKAFNNAGEGVPLYESATTRSITDPTDPVDYYPLLDDFPTSGPDVSTPMLPPVGVQAVALTHEAVRVSWADNSVPKNQKTSDVRLYTVRWRTSFSASAKYKSEDTTSLSYTATGLKPNTMYEFSVMVTKNRRSSTWSMTAHATTYEAAPTSAPKDLTVITREGKPRAVIVSWQPPLEANGKITAYILFYTLDKNIPIDDWIMETISGDRLTHQIMDLSLDTMYYFRIQARNVKGVGPLSDPILFRTLKVEHPDKMANDQGRHGDGGYWPVDTNLIDRSTLNEPPIGQMHPPHGSVTPQKNSNLLVITVVTVGVLTVLVVVIVAVICTRRSSAQQRKKRATHSVSKRKGSQKDLRPPDLWIHHEEMEMKNIEKPTGTDPAGRDSPIQSCQDLTPVSHSQSETQMGSKSASHSGQDTEDAGSSMSTLERSLAARRATRAKLMIPMEAQSSNPAVVSAIPVPTLESAQYPGILPSPTCGYPHPQFTLRPVPFPTLSVDRGFGAGRTQSVSEGPTTQQQPMLPPAQPEHPSSEEAPSRTIPTACVRPTHPLRSFANPLLPPPMSAIEPKVPYTPLLSQPGPTLPKTHVKTASLGLAGKARSPLLPVSVPTAPEVSEESHKPTEDPASVYEQDDLSEQMASLEGLMKQLNAITGSAF

SEQ ID NO: 6 - protein, 772 aa, Homo sapiens
SGVPVIKWKKDGIHLALGMDERKQQLSNGSLLIQNILHSRHHKPDEGLYQCEASLGDSGSIISRTAKVAVAGPLRFLSQTESVTAFMGDTVLLKCEVIGEPMPTIHWQKNQQDLTPIPGDSRVVVLPSGALQISRLQPGDIGIYRCSARNPASSRTGNEAEVRILSDPGLHRQLYFLQRPSNVVAIEGKDAVLEGCVSGYPPPSFTWLRGEEVIQLRSKKYSLLGGSNLLISNVTDDDSGMYTCVVTYKNENISASAELTVLVPPWFLNHPSNLYAYESMDIEFECTVSGKPVPTVNWMKNGDVVIPSDYFQIVGGSNLRILGVVKSDEGFYQCVAENEAGNAQTSAQLIVPKPAIPSSSVLPSAPRDVVPVLVSSRFVRLSWRPPAEAKGNIQTFTVFFSREGDNRERALNTTQPGSLQLTVGNLKPEAMYTFRVVAYNEWGPGESSQPIKVATQPELQVPGPVENLQAVSTSPTSILITWEPPAYANGPVQGYRLFCTEVSTGKEQNIEVDGLSYKLEGLKKFTEYSLRFLAYNRYGPGVSTDDITVVTLSDVPSAPPQNVSLEVVNSRSIKVSWLPPPSGTQNGFITGYKIRHRKTTRRGEMETLEPNNLWYLFTGLEKGSQYSFQVSAMTVNGTGPPSNWYTAETPENDLDESQVPDQPSSLHVRPQTNCIIMSWTPPLNPNIVVRGYIIGYGVGSPYAETVRVDSKQRYYSIERLESSSHYVISLKAFNNAGEGVPLYESATTRSITVRRHNISKLHSNRPQTKHNV

SEQ ID NO: 7 - protein, 945 aa, Mus musculus
MRARSGVRSALLLALLLCWDPTPSLAGVDSAGQVLPDSYPSAPAEQLPYFLLEPQDAYIVKNKPVELHCRAFPATQIYFKCNGEWVSQNDHVTQESLDEATGLRVREVQIEVSRQQVEELFGLEDYWCQCVAWSSSGTTKSRRAYIRIAYLRKNFDQEPLAKEVPLDHEVLLQCRPPEGVPVAEVEWLKNEDVIDPAQDTNFLLTIDHNLIIRQARLSDTANYTCVAKNIVAKRRSTTATVIVYVNGGWSSWAEWSPCSNRCGRGWQKRTRTCTNPAPLNGGAFCEGQAFQKTACTTVCPVDGAWTEWSKWSACSTECAHWRSRECMAPPPQNGGRDCSGTLLDSKNCTDGLCVLNQRTLNDPKSHPLETSGDVALYAGLVVAVFVVVAVLMAVGVIVYRRNCRDFDTDITDSSAALTGGFHPVNFKTARPNNPQLLHPSAPPDLTASAGIYRGPVYALQDSADKIPMTNSPLLDPLPSLKIKVYNSSTIGSGSGLADGADLLGVLPPGTYPGDFSRDTHFLHLRSASLGSQHLLGLPRDPSSSVSGTFGCLGGRLSLPGTGVSLLVPNGAIPQGKFYDLYLHINKAESTLPLSEGSQTVLSPSVTCGPTGLLLCRPVVLTVPHCAEVIAGDWIFQLKTQAHQGHWEEVVTLDEETLNTPCYCQLEAKSCHILLDQLGTYVFMGESYSRSAVKRLQLAIFAPALCTSLEYSLRVYCLEDTPVALKEVLELERTLGGYLVEEPKPLLFKDSYHNLRLSLHDIPHAHWRSKLLAKYQEIPFYHVWNGSQRALHCTFTLERHSLASTEFTCKVCVRQVEGEGQIFQLHTTLAETPAGSLDALCSAPGNAITTQLGPYAFKIPLSIRQKICSSLDAPNSRGNDWRLLAQKLSMDRYLNYFATKASPTGVILDLWEARQQDDGDLNSLASALEEMGKSEMLVAMATDGDC

SEQ ID NO: 8 - protein, 11 aa, Mus musculus
MVTKNRRSSTW

SEQ ID NO: 9 - protein, 6 aa, Homo sapiens
HARRCR

SEQ ID NO: 10 - protein, 7 aa, Homo sapiens
MELYKLS

SEQ ID NO: 11 - protein, 4 aa, artificial
  portion of NET1 binding domain
  misc_feature (2)..(2): Xaa can be any naturally occurring amino acid
KXRR

SEQ ID NO: 12 - protein, 12 aa, artificial
  NET1 binding domain
  misc_feature (5)..(5): Xaa can be any naturally occurring amino acid
MVTKXRRSSTWS

SEQ ID NO: 13 - protein, 8 aa, Homo sapiens
RRCRFNME

SEQ ID NO: 14 - protein, 8 aa, Homo sapiens
DTCHFDVN

SEQ ID NO: 15 - protein, 4 aa, Homo sapiens
KGRR

SEQ ID NO: 16 - protein, 28 aa, Homo sapiens
CNLHARRCRFNMELYKLSGRKSGGVCLN

SEQ ID NO: 17 - protein, 64 aa, artificial
  V-2 region
  misc_feature (47)..(47): Xaa can be any naturally occurring amino acid
CNCNLHARRCRFNMELYKLSGRKSGGVCLNCRHNTAGRHCHYCKEGXYRDMGKPITHRKACKAC

SEQ ID NO: 18 - protein, 101 aa, Mus musculus
MMPPVGVQASILSHDTIRITWADNSLPKHQKITDSRYYTVRWKTNIPANTKYKNANATTLSYLVTGLKPNTLYEFSVMVTKGRRSSTWSMTAHGATFELVP

SEQ ID NO: 19 - protein, 101 aa, Mus musculus
MLPPVGVQAVALTHEAVRVSWADNSVPKNQKTSDVRLYTVRWRTSFSASAKYKSEDTTSLSYTATGLKPNTMYEFSVMVTKNRRSSTWSMTAHATTYEAAP

SEQ ID NO: 20 - protein, 284 aa, Mus musculus
MMRAVWEALAALAAVACLVGAVRGGPGLSMFAGQAAQPDPCSDENGHPRRCIPDFVNAAFGKDVRVSSTCGRPPARYCVVSERGEERLRSCHLCNSSDPKKAHPPAFLTDLNNPHNLTCWQSENYLQFPHNVTLTLSLGKKFEVTYVSLQFCSPRPESMAIYKSMDYGRTWVPFQFYSTQCRKMYNRPHRAPITKQNEQEAVCTDSHTDMRPLSGGLIAFSTLDGRPSAHDFDNSPVLQDWVTATDIRVAFSRLHTFGDENEDDSELARDSYYYAVSDLQVGGR

SEQ ID NO: 21 - protein, 249 aa, Mus musculus
QEPEFSYGCAEGSCYPATGDLLIGRAQKLSVTSTCGLHKPEPYCIVSHLQEDKKCFICDSRDPYHETLNPDSHLIENVVTTFAPNRLKIWWQSENGVENVTIQLDLEAEFHFTHLIMTFKTFRPAAMLIERSSDFGKAWGVYRYFAYDCESSFPGISTGPMKKVDDIICDSRYSDIEPSTEGEVIFRALDPAFKIEDPYSPRIQNLLKITNLRIKFVKLHTLGDNLLDSRMEIREKYYYAVYDMVVRGN

SEQ ID NO: 22 - protein, 252 aa, Mus musculus
PYFNLAEGARITASATCGEEAPTRSVSRPTEDLYCKLVGGPVAGGDPNQTIQGQYCDICTAANSNKAHPVSNAIDGTERWWQSPPLSRGLEYNEVNVTLDLGQVFHVAYVLIKFANSPRPDLWVLERSTDFGHTYQPWQFFASSKRDCLERFGPRTLERITQDDDVICTTEYSRIVPLENGEIVVSLVNGRPGALNFSYSPLLRDFTKATNIRLRFLRTNTLLGHLMGKALRDPTVTRRYYYSIKDISIGGR

SEQ ID NO: 23 - protein, 251 aa, Mus musculus
AAMDECADEGGRPQRCMPEFVNAAFNVTVVATNTCGTPPEEYCVQTGVTGVTKSCHLCDAGQQHLQHGAAFLTDYNNQADTTWWQSQTMLAGVQYPNSINLTLHLGKAFDITYVRLKFHTSRPESFAIYKRTREDGPWIPYQYYSGSCENTYSKANRGFIRTGGDEQQALCTDEFSDISPLTGGNVAFSTLEGRPSAYNFDNSPVLQEWVTATDIRVTLNRLNTFGDEVFNDPKVLKSYYYAISDFAVGGR

SEQ ID NO: 24 - protein, 63 aa, Mus musculus
CNCNLHARRCRFNMELYKLSGRKSGGVCLNCRHNTAGRHCHYCKEGFYRDMGKPITHRKACKA

SEQ ID NO: 25 - protein, 63 aa, Mus musculus
CSCNGHARRCRFNMELYRLSGRRSGGVCLNCRHNTAGRHCHYCREGFYRDPGRVLSDRRACRA

SEQ ID NO: 26 - protein, 63 aa, Mus musculus
CKCNGHADTCHFDVNVWEASGNRSGGVCNNCQHNTEGQHCQRCKPGFYRDLRRPFSAPDACKA

SEQ ID NO: 27 - protein, 63 aa, Mus musculus
CNCNEHSSSCHFDMAVFLATGNVSGGVCDNCQHNTMGRNCEQCKPFYFQHPERDIRDPNLCEP

SEQ ID NO: 28 - protein, 70 aa, Mus musculus
CNCHGHAYDCYYDPEVDRRNASQNQDNVYQGGGVCLDCQHHTTGINCERCLPGFFRAPDQPLDSPHVCRP

SEQ ID NO: 29 - protein, 56 aa, Mus musculus
CDCNGRSQECYFDPELYRSTGHGGHCTNCRDNTDGAKCERCRENFFRLGNTEACSP

SEQ ID NO: 30 - protein, 90 aa, Mus musculus
VQASILSHDTIRITWADNSLPKHQKITDSRYYTVRWKTNIPANTKYKNANATTLSYLVTGLKPNTLYEFSVMVTKGRRSSTWSMTAHGAT

SEQ ID NO: 31 - protein, 90 aa, Mus musculus
VQAVALTHEAVRVSWADNSVPKNQKTSDVRLYTVRWRTSFSASAKYKSEDTTSLSYTATGLKPNTMYEFSVMVTKNRRSSTWSMTAHATT